(19) United States
(12) Patent Application Publication
(10) Pub. No.: US 2023/0066798 A1
(43) Pub. Date: Mar. 2, 2023

(54) COMPOSITIONS AND METHODS FOR POST-OPERATIVE OCULAR CARE

(71) Applicant: Ocular Science, Inc., Manhattan Beach, CA (US)

(72) Inventors: Anthony Sampietro, Hermosa Beach, CA (US); Damien Goldberg, Manhattan Beach, CA (US); Amy Frost, Stevensville, MT (US); Brian Holdorf, Missoula, MT (US); Francis Mah, Del Mar, CA (US)

(21) Appl. No.: 17/983,388

(22) Filed: Nov. 8, 2022

Related U.S. Application Data
(63) Continuation of application No. 17/222,419, filed on Apr. 5, 2021.
(60) Provisional application No. 63/037,171, filed on Jun. 10, 2020.

(51) Int. Cl. A61K 31/573; A61K 9/00; A61K 31/196; A61K 31/4709

(52) U.S. Cl. CPC A61K 31/573; A61K 9/0048; A61K 31/196; A61K 31/4709

(57) ABSTRACT

Provided herein are certain preservative-free ophthalmic formulations for post-operative ocular care.

### CROSS-REFERENCE

This application is a continuation of U.S. application Ser. No. 17/222,419, filed Apr. 5, 2021, which claims the benefit of U.S. Provisional Application No. 63/037,171, filed Jun. 10, 2020, in which each application is incorporated herein by reference.

### BACKGROUND OF THE INVENTION

Post-operative care for ocular surgeries involves the administration of several medications. These medications are typically administered in the form of individual bottles of eye drops containing one active pharmaceutical ingredient (API) each, resulting in poor patient compliance due to the high number of dropper bottles, increased patient cost, and increased ocular exposure to preservatives. Consequently, there is a need for the development of preservative-free ocular formulations that contain multiple APIs in a single dropper bottle.

### SUMMARY OF THE INVENTION

Provided herein, in one aspect, is a method for treating an ocular condition of an eye, comprising administering a pharmaceutical composition at, in, or around the eye via a delivery device and per a predetermined dosing regimen, wherein:

the pharmaceutical composition is free of preservatives;

the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;
   wherein these percentages are with respect to weight per volume; and
the ocular condition is care after cataract surgery, care after LASIK surgery, care for a retina of the eye after cataract surgery, care for a retina of the eye after retina surgery, in preparation for an intraocular procedure, or during the intraocular procedure.

In some embodiments, the delivery device is an eye dropper. In some embodiments, the eye dropper is a multidose eye dropper. In some embodiments, the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold.

In some embodiments, the predetermined dosing regimen is once per day, twice per day, three times per day, once every other day, once per week, once every other week, or once monthly.

In some embodiments, the pharmaceutical composition comprises prednisolone PO₄ about 1%, moxifloxacin HCl about 0.5%, and bromfenac about 0.075%.

In some embodiments, the pharmaceutical composition comprises prednisolone PO₄ about 1% and moxifloxacin HCl about 0.5%.

In some embodiments, the pharmaceutical composition comprises moxifloxacin HCl about 0.5% and bromfenac about 0.075%.

In some embodiments, the pharmaceutical composition comprises difluprednate about 0.05%, moxifloxacin HCl about 0.5%, and bromfenac about 0.075%.

Provided herein, in another aspect, is a method for treating an ocular condition of an eye, comprising administering a pharmaceutical composition at, in, or around the eye via a delivery device and per a predetermined dosing regimen; wherein the pharmaceutical composition comprises at least two active pharmaceutical ingredients compounded and stored in communication with each other; wherein the pharmaceutical composition is free of preservatives; wherein the method is more effective as compared against a preexisting method; wherein the preexisting method administers the at least two active pharmaceutical ingredients from at least two separate and different containers; and wherein the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%; wherein these percentages are with respect to weight per volume.

In some embodiments, the ocular condition is one or more of: care after cataract surgery, care after LASIK surgery, care for a retina of the eye after cataract surgery, care for a retina of the eye after retina surgery, in preparation for an intraocular procure, or during an intraocular procedure.

In some embodiments, the delivery device is an eye dropper. In some embodiments, the eye dropper is a multidose eye dropper. In some embodiments, the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold.

In some embodiments, the predetermined dosing regimen is once per day, twice per day, three times per day, once every other day, once per week, once every other week, or once monthly.

In some embodiments, the pharmaceutical composition comprises prednisolone PO₄ about 1%, moxifloxacin HCl about 0.5%, and bromfenac about 0.075%.

In some embodiments, the pharmaceutical composition comprises prednisolone PO₄ about 1% and moxifloxacin HCl about 0.5%.

In some embodiments, the pharmaceutical composition comprises moxifloxacin HCl about 0.5% and bromfenac about 0.075%.

In some embodiments, the pharmaceutical composition comprises difluprednate about 0.05%, moxifloxacin HCl about 0.5%, and bromfenac about 0.075%.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a flow diagram of a method for compounding a given pharmaceutical composition.

### DETAILED DESCRIPTION OF THE INVENTION

Recognized herein is the need for the development of preservative-free ocular formulations that contain multiple APIs in a single dropper bottle for the purposes of providing post-operative ocular care.

Provided herein, in one aspect, is a method for treating an ocular condition of an eye, comprising administering a pharmaceutical composition at, in, or around the eye via a delivery device and per a predetermined dosing regimen, wherein:

the pharmaceutical composition is free of preservatives;

the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;
   wherein these percentages are with respect to weight per volume; and
the ocular condition is care after cataract surgery, care after LASIK surgery, care for a retina of the eye after cataract surgery, care for a retina of the eye after retina surgery, in preparation for an intraocular procedure, or during the intraocular procedure.

### Pharmaceutical Composition 1: Prednisolone PO₄ 1%/Moxifloxacin HCl 0.5%/Bromfenac 0.075%

In some embodiments, the pharmaceutical composition comprises prednisolone PO₄ about 1%, moxifloxacin HCl about 0.5%, and bromfenac about 0.075%.

In some embodiments, the pharmaceutical composition comprises prednisolone or a pharmaceutically acceptable salt or analog thereof. In some embodiments, the pharmaceutical composition comprises prednisolone. In some embodiments, the pharmaceutical composition comprises prednisolone sodium phosphate (prednisolone PO₄). In some embodiments, the pharmaceutical composition comprises prednisolone acetate. In some embodiments, the pharmaceutical composition comprises prednisolone hemisuccinate sodium salt. In some embodiments, the pharmaceutical composition comprises prednisolone hemisuccinate. In some embodiments, the pharmaceutical composition comprises prednisolone hexanoate. In some embodiments, the pharmaceutical composition comprises prednisolone pivalate. In some embodiments, the pharmaceutical composition comprises prednisolone tebutate.

In some embodiments, prednisolone PO₄ may be known as prednisolone, prednisolone phosphate, prednisolone NaPO₄, or prednisolone sodium phosphate. In some embodiments, prednisolone PO₄ may be prepared to meet USP monograph for prednisolone sodium phosphate ophthalmic solution. In some embodiments, prednisolone PO₄ may be an anti-inflammatory steroid. A mechanism of action for prednisolone PO₄ may be inhibition of migration of polymorphonuclear leukocytes and capilla increase reversal. In some embodiments, prednisolone PO₄ may be used for treating inflammation in the eye, at the eye, and/or around the eye via use of eye drops. In some embodiments, prednisolone PO₄ may be used in preparation for ocular surgery. In some embodiments, prednisolone PO₄ may be used during ocular surgery. In some embodiments, prednisolone PO₄ may be used after ocular surgery.

In some embodiments, the pharmaceutical composition comprises prednisolone in an amount of about 1% with respect to weight per volume. In some embodiments, the pharmaceutical composition comprises from 0.9% to 1.1% prednisolone. In some embodiments, the pharmaceutical composition comprises from about 0.9% to about 1.0% prednisolone. In some embodiments, the pharmaceutical composition comprises from about 1.0% to about 1.1% prednisolone. In some embodiments, the pharmaceutical composition comprises from about 0.90% to about 0.95% prednisolone. In some embodiments, the pharmaceutical composition comprises from about 0.95% to about 1.00% prednisolone. In some embodiments, the pharmaceutical composition comprises from about 1.00% to about 1.05% prednisolone. In some embodiments, the pharmaceutical composition comprises from about 1.05% to about 1.10% prednisolone. In some embodiments, the pharmaceutical composition comprises 0.90% prednisolone. In some embodiments, the pharmaceutical composition comprises 0.91% prednisolone. In some embodiments, the pharmaceutical composition comprises 0.92% prednisolone. In some embodiments, the pharmaceutical composition comprises 0.93% prednisolone. In some embodiments, the pharmaceutical composition comprises 0.94% prednisolone. In some embodiments, the pharmaceutical composition comprises 0.95% prednisolone. In some embodiments, the pharmaceutical composition comprises 0.96% prednisolone. In some embodiments, the pharmaceutical composition comprises 0.97% prednisolone. In some embodiments, the pharmaceutical composition comprises 0.98% prednisolone. In some embodiments, the pharmaceutical composition comprises 0.99% prednisolone. In some embodiments, the pharmaceutical composition comprises 1.00% prednisolone. In some embodiments, the pharmaceutical composition comprises 1.01% prednisolone. In some embodiments, the pharmaceutical composition comprises 1.02% prednisolone. In some embodiments, the pharmaceutical composition comprises 1.03% prednisolone. In some embodiments, the pharmaceutical composition comprises 1.04% prednisolone. In some embodiments, the pharmaceutical composition comprises 1.05% prednisolone. In some embodiments, the pharmaceutical composition comprises 1.06% prednisolone. In some embodiments, the pharmaceutical composition comprises 1.07% prednisolone. In some embodiments, the pharmaceutical composition comprises 1.08% prednisolone. In some embodiments, the pharmaceutical composition comprises 1.09% prednisolone. In some embodiments, the pharmaceutical composition comprises 1.10% prednisolone.

In some embodiments, the pharmaceutical composition comprises moxifloxacin or a pharmaceutically acceptable salt thereof. In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the pharmaceutical composition comprises moxifloxacin in an amount of about 0.5% with respect to weight per volume. In some embodiments, the pharmaceutical composition comprises from 0.45% to 0.55% moxifloxacin. In some embodiments, the pharmaceutical composition comprises from 0.45% to 0.50% moxifloxacin. In some embodiments, the pharmaceutical composition comprises from 0.50% to 0.55% moxifloxacin. In some embodiments, the pharmaceutical composition comprises 0.45% moxifloxacin. In some embodiments, the pharmaceutical composition comprises 0.46% moxifloxacin. In some embodiments, the pharmaceutical composition comprises 0.47% moxifloxacin. In some embodiments, the pharmaceutical composition comprises 0.48% moxifloxacin. In some embodiments, the pharmaceutical composition comprises 0.49% moxifloxacin. In some embodiments, the pharmaceutical composition comprises 0.50% moxifloxacin. In some embodiments, the pharmaceutical composition comprises 0.51% moxifloxacin. In some embodiments, the pharmaceutical composition comprises 0.52% moxifloxacin. In some embodiments, the pharmaceutical composition comprises 0.53% moxifloxacin. In some embodiments, the pharmaceutical composition comprises 0.54% moxifloxacin. In some embodiments, the pharmaceutical composition comprises 0.55% moxifloxacin.

In some embodiments, the pharmaceutical composition comprises bromfenac or a pharmaceutically acceptable salt thereof. In some embodiments, bromfenac may be known as bromfenac, bromfenac sodium, and bromfenac ophthalmic solution. In some embodiments, the bromfenac may be known as bromfenac sodium sesquihydrate. In some embodiments, the bromfenac may be a non-steroidal anti-inflammatory drug (NSAID). In some embodiments, bromfenac may block prostaglandin synthesis through cyclooxygenase inhibition, demonstrating COX-2 preference with a lesser affinity for COX-1. In some embodiments, bromfenac may be used as an analgesic. In some embodiments, bromfenac may be used to treat ocular pain. In some embodiments, bromfenac may be used to treat ocular inflammation. In some embodiments, bromfenac may be used to treat, promote, and/or facilitate post eye surgery healing and/or health.

In some embodiments, the pharmaceutical composition comprises bromfenac in an amount of about 0.075% with respect to weight per volume. In some embodiments, the pharmaceutical composition comprises from 0.0675% to 0.0825% bromfenac. In some embodiments, the pharmaceutical composition comprises from 0.0675% to 0.0700% bromfenac. In some embodiments, the pharmaceutical composition comprises from 0.0700% to 0.0725% bromfenac. In some embodiments, the pharmaceutical composition comprises from 0.0725% to 0.0750% bromfenac. In some embodiments, the pharmaceutical composition comprises from 0.0750% to 0.0775% bromfenac. In some embodiments, the pharmaceutical composition comprises from 0.0775% to 0.0800% bromfenac. In some embodiments, the pharmaceutical composition comprises from 0.0800% to 0.0825% bromfenac. In some embodiments, the pharmaceutical composition comprises 0.0675% bromfenac. In some embodiments, the pharmaceutical composition comprises 0.0680% bromfenac. In some embodiments, the pharmaceutical composition comprises 0.0685% bromfenac. In some embodiments, the pharmaceutical composition comprises 0.0690% bromfenac. In some embodiments, the pharmaceutical composition comprises 0.0695% bromfenac. In some embodiments, the pharmaceutical composition comprises 0.0700% bromfenac. In some embodiments, the pharmaceutical composition comprises 0.0705% bromfenac. In some embodiments, the pharmaceutical composition comprises 0.0710% bromfenac. In some embodiments, the pharmaceutical composition comprises 0.0715% bromfenac. In some embodiments, the pharmaceutical composition comprises 0.0720% bromfenac. In some embodiments, the pharmaceutical composition comprises 0.0725% bromfenac. In some embodiments, the pharmaceutical composition comprises 0.0730% bromfenac. In some embodiments, the pharmaceutical composition comprises 0.0735% bromfenac. In some embodiments, the pharmaceutical composition comprises 0.0740% bromfenac. In some embodiments, the pharmaceutical composition comprises 0.0745% bromfenac. In some embodiments, the pharmaceutical composition comprises 0.0750% bromfenac. In some embodiments, the pharmaceutical composition comprises 0.0755% bromfenac. In some embodiments, the pharmaceutical composition comprises 0.0760% bromfenac. In some embodiments, the pharmaceutical composition comprises 0.0765% bromfenac. In some embodiments, the pharmaceutical composition comprises 0.0770% bromfenac. In some embodiments, the pharmaceutical composition comprises 0.0775% bromfenac. In some embodiments, the pharmaceutical composition comprises 0.0780% bromfenac. In some embodiments, the pharmaceutical composition comprises 0.0785% bromfenac. In some embodiments, the pharmaceutical composition comprises 0.0790% bromfenac. In some embodiments, the pharmaceutical composition comprises 0.0795% bromfenac. In some embodiments, the pharmaceutical composition comprises 0.0800% bromfenac. In some embodiments, the pharmaceutical composition comprises 0.0805% bromfenac. In some embodiments, the pharmaceutical composition comprises 0.0810% bromfenac. In some embodiments, the pharmaceutical composition comprises 0.0815% bromfenac. In some embodiments, the pharmaceutical composition comprises 0.0820% bromfenac. In some embodiments, the pharmaceutical composition comprises 0.0825% bromfenac.

In some embodiments, the pH of the pharmaceutical composition is about 8. In some embodiments, the pH of the pharmaceutical composition is greater than 8. In some embodiments, the pH of the pharmaceutical composition is 7.0. In some embodiments, the pH of the pharmaceutical composition is 7.1. In some embodiments, the pH of the pharmaceutical composition is 7.2. In some embodiments, the pH of the pharmaceutical composition is 7.3. In some embodiments, the pH of the pharmaceutical composition is 7.4. In some embodiments, the pH of the pharmaceutical composition is 7.5. In some embodiments, the pH of the pharmaceutical composition is 7.6. In some embodiments, the pH of the pharmaceutical composition is 7.7. In some embodiments, the pH of the pharmaceutical composition is 7.8. In some embodiments, the pH of the pharmaceutical composition is 7.9. In some embodiments, the pH of the pharmaceutical composition is 8.0. In some embodiments, the pH of the pharmaceutical composition is 8.1. In some embodiments, the pH of the pharmaceutical composition is 8.2. In some embodiments, the pH of the pharmaceutical composition is 8.3. In some embodiments, the pH of the pharmaceutical composition is 8.4. In some embodiments, the pH of the pharmaceutical composition is 8.5. In some embodiments, the pH of the pharmaceutical composition is 8.6. In some embodiments, the pH of the pharmaceutical composition is 8.7. In some embodiments, the pH of the pharmaceutical composition is 8.8. In some embodiments, the pH of the pharmaceutical composition is 8.9. In some embodiments, the pH of the pharmaceutical composition is 9.0.

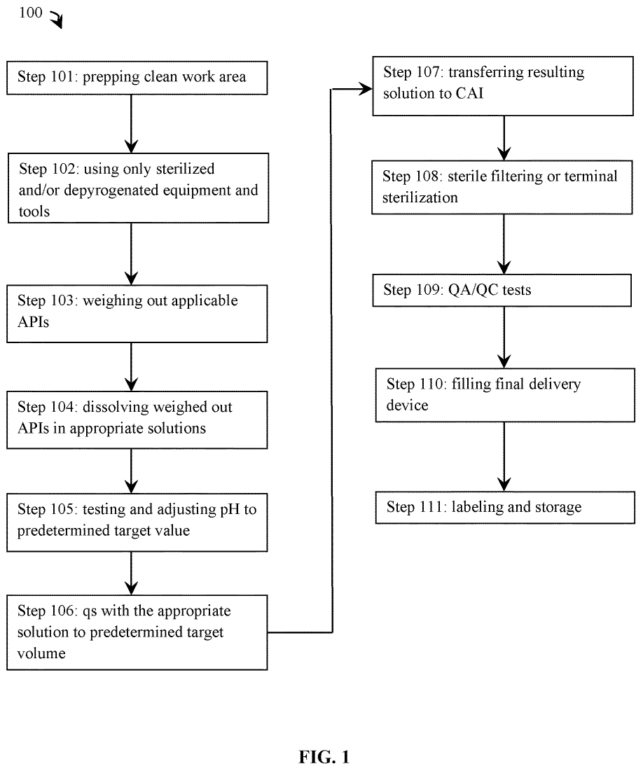

In some embodiments, compounding the pharmaceutical composition comprising 1% Prednisolone PO₄, 0.5% Moxifloxacin HCl, and 0.075% Bromfenac may comprise steps of: (step 101) prepping clean work area (e.g., cleaning and/or disinfecting); (step 102) using only sterilized and/or depyrogenated equipment; (step 103) weighing applicable APIs (e.g., Prednisolone PO₄, Moxifloxacin HCl, and Bromfenac) in a powder hood (with the 1%, 0.5%, and 0.075% targets in mind); (step 104) dissolving weighed out API powders in sterile water (or SWFI) (with the 1%, 0.5%, and 0.075% targets in mind); (step 105) testing and adjusting the pH to a target of >8 via use of sodium hydroxide and pH meter (calibrated); (step 106) qs (“quantity sufficient”) with the sterile water (or SWFI) with the 1%, 0.5%, and 0.075% targets in mind; (step 107) transferring resulting solution to a compounding aseptic isolator (CAI); (step 108) sterile filtering (e.g., a 0.22 micron filter) the resulting solution to yield the pharmaceutical composition comprising 1% Prednisolone PO₄, 0.5% Moxifloxacin HCl, and 0.075% Bromfenac; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant.

### Pharmaceutical Composition 2: Prednisolone PO₄ 1%/Moxifloxacin HCl 0.5%

In some embodiments, the pharmaceutical composition comprises prednisolone PO₄ about 1% and moxifloxacin HCl about 0.5%.

In some embodiments, the pharmaceutical composition comprises prednisolone or a pharmaceutically acceptable salt or analog thereof. In some embodiments, the pharmaceutical composition comprises prednisolone. In some embodiments, the pharmaceutical composition comprises prednisolone sodium phosphate (prednisolone PO₄). In some embodiments, the pharmaceutical composition comprises prednisolone acetate. In some embodiments, the pharmaceutical composition comprises prednisolone hemisuccinate sodium salt. In some embodiments, the pharmaceutical composition comprises prednisolone hemisuccinate. In some embodiments, the pharmaceutical composition comprises prednisolone hexanoate. In some embodiments, the pharmaceutical composition comprises prednisolone pivalate. In some embodiments, the pharmaceutical composition comprises prednisolone tebutate.

In some embodiments, prednisolone PO₄ may be known as prednisolone, prednisolone phosphate, prednisolone NaPO₄, or prednisolone sodium phosphate. In some embodiments, prednisolone PO₄ may be prepared to meet USP monograph for prednisolone sodium phosphate ophthalmic solution. In some embodiments, prednisolone PO₄ may be an anti-inflammatory steroid. A mechanism of action for prednisolone PO₄ may be inhibition of migration of polymorphonuclear leukocytes and capilla increase reversal. In some embodiments, prednisolone PO₄ may be used for treating inflammation in the eye, at the eye, and/or around the eye via use of eye drops. In some embodiments, prednisolone PO₄ may be used in preparation for ocular surgery. In some embodiments, prednisolone PO₄ may be used during ocular surgery. In some embodiments, prednisolone PO₄ may be used after ocular surgery.

In some embodiments, the pharmaceutical composition comprises prednisolone in an amount of about 1% with respect to weight per volume. In some embodiments, the pharmaceutical composition comprises from 0.9% to 1.1% prednisolone. In some embodiments, the pharmaceutical composition comprises from about 0.9% to about 1.0% prednisolone. In some embodiments, the pharmaceutical composition comprises from about 1.0% to about 1.1% prednisolone. In some embodiments, the pharmaceutical composition comprises from about 0.90% to about 0.95% prednisolone. In some embodiments, the pharmaceutical composition comprises from about 0.95% to about 1.00% prednisolone. In some embodiments, the pharmaceutical composition comprises from about 1.00% to about 1.05% prednisolone. In some embodiments, the pharmaceutical composition comprises from about 1.05% to about 1.10% prednisolone. In some embodiments, the pharmaceutical composition comprises 0.90% prednisolone. In some embodiments, the pharmaceutical composition comprises 0.91% prednisolone. In some embodiments, the pharmaceutical composition comprises 0.92% prednisolone. In some embodiments, the pharmaceutical composition comprises 0.93% prednisolone. In some embodiments, the pharmaceutical composition comprises 0.94% prednisolone. In some embodiments, the pharmaceutical composition comprises 0.95% prednisolone. In some embodiments, the pharmaceutical composition comprises 0.96% prednisolone. In some embodiments, the pharmaceutical composition comprises 0.97% prednisolone. In some embodiments, the pharmaceutical composition comprises 0.98% prednisolone. In some embodiments, the pharmaceutical composition comprises 0.99% prednisolone. In some embodiments, the pharmaceutical composition comprises 1.00% prednisolone. In some embodiments, the pharmaceutical composition comprises 1.01% prednisolone. In some embodiments, the pharmaceutical composition comprises 1.02% prednisolone. In some embodiments, the pharmaceutical composition comprises 1.03% prednisolone. In some embodiments, the pharmaceutical composition comprises 1.04% prednisolone. In some embodiments, the pharmaceutical composition comprises 1.05% prednisolone. In some embodiments, the pharmaceutical composition comprises 1.06% prednisolone. In some embodiments, the pharmaceutical composition comprises 1.07% prednisolone. In some embodiments, the pharmaceutical composition comprises 1.08% prednisolone. In some embodiments, the pharmaceutical composition comprises 1.09% prednisolone. In some embodiments, the pharmaceutical composition comprises 1.10% prednisolone.

In some embodiments, the pharmaceutical composition comprises moxifloxacin or a pharmaceutically acceptable salt thereof. In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the pharmaceutical composition comprises moxifloxacin in an amount of about 0.5% with respect to weight per volume. In some embodiments, the pharmaceutical composition comprises from 0.45% to 0.55% moxifloxacin. In some embodiments, the pharmaceutical composition comprises from 0.45% to 0.50% moxifloxacin. In some embodiments, the pharmaceutical composition comprises from 0.50% to 0.55% moxifloxacin. In some embodiments, the pharmaceutical composition comprises 0.45% moxifloxacin. In some embodiments, the pharmaceutical composition comprises 0.46% moxifloxacin. In some embodiments, the pharmaceutical composition comprises 0.47% moxifloxacin. In some embodiments, the pharmaceutical composition comprises 0.48% moxifloxacin. In some embodiments, the pharmaceutical composition comprises 0.49% moxifloxacin. In some embodiments, the pharmaceutical composition comprises 0.50% moxifloxacin. In some embodiments, the pharmaceutical composition comprises 0.51% moxifloxacin. In some embodiments, the pharmaceutical composition comprises 0.52% moxifloxacin. In some embodiments, the pharmaceutical composition comprises 0.53% moxifloxacin. In some embodiments, the pharmaceutical composition comprises 0.54% moxifloxacin. In some embodiments, the pharmaceutical composition comprises 0.55% moxifloxacin.

In some embodiments, the pH of the pharmaceutical composition is about 7. In some embodiments, the pH of the pharmaceutical composition is greater than 7. In some embodiments, the pH of the pharmaceutical composition is 7.0. In some embodiments, the pH of the pharmaceutical composition is 7.1. In some embodiments, the pH of the pharmaceutical composition is 7.2. In some embodiments, the pH of the pharmaceutical composition is 7.3. In some embodiments, the pH of the pharmaceutical composition is 7.4. In some embodiments, the pH of the pharmaceutical composition is 7.5. In some embodiments, the pH of the pharmaceutical composition is 7.6. In some embodiments, the pH of the pharmaceutical composition is 7.7. In some embodiments, the pH of the pharmaceutical composition is 7.8. In some embodiments, the pH of the pharmaceutical composition is 7.9. In some embodiments, the pH of the pharmaceutical composition is 8.0.

In some embodiments, compounding the pharmaceutical composition comprising 1% Prednisolone PO₄ and 0.5% Moxifloxacin HCl may comprise steps of: (step 101) prepping clean work area (e.g., cleaning and/or disinfecting); (step 102) using only sterilized and/or depyrogenated equipment; (step 103) weighing applicable APIs (e.g., Prednisolone PO₄ and Moxifloxacin HCl) in a powder hood (with the 1% and 0.5% targets in mind); (step 104) dissolving weighed out API powders in sterile water (or SWFI) (with the 1% and 0.5% targets in mind); (step 105) testing and adjusting the pH to a target of >7 via use of sodium hydroxide and pH meter (calibrated); (step 106) qs (“quantity sufficient”) with the sterile water (or SWFI) with the 1% and 0.5% targets in mind; (step 107) transferring resulting solution to a compounding aseptic isolator (CAI); (step 108) sterile filtering (e.g., a 0.22 micron filter) the resulting solution to yield the pharmaceutical composition comprising 1% Prednisolone PO₄ and 0.5% Moxifloxacin HCl; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant.

### Pharmaceutical Composition 3: Moxifloxacin HCl 0.5%/Bromfenac 0.075%

In some embodiments, the pharmaceutical composition comprises moxifloxacin HCl about 0.5% and bromfenac about 0.075%.

In some embodiments, the pharmaceutical composition comprises moxifloxacin or a pharmaceutically acceptable salt thereof. In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the pharmaceutical composition comprises moxifloxacin in an amount of about 0.5% with respect to weight per volume. In some embodiments, the pharmaceutical composition comprises from 0.45% to 0.55% moxifloxacin. In some embodiments, the pharmaceutical composition comprises from 0.45% to 0.50% moxifloxacin. In some embodiments, the pharmaceutical composition comprises from 0.50% to 0.55% moxifloxacin. In some embodiments, the pharmaceutical composition comprises 0.45% moxifloxacin. In some embodiments, the pharmaceutical composition comprises 0.46% moxifloxacin. In some embodiments, the pharmaceutical composition comprises 0.47% moxifloxacin. In some embodiments, the pharmaceutical composition comprises 0.48% moxifloxacin. In some embodiments, the pharmaceutical composition comprises 0.49% moxifloxacin. In some embodiments, the pharmaceutical composition comprises 0.50% moxifloxacin. In some embodiments, the pharmaceutical composition comprises 0.51% moxifloxacin. In some embodiments, the pharmaceutical composition comprises 0.52% moxifloxacin. In some embodiments, the pharmaceutical composition comprises 0.53% moxifloxacin. In some embodiments, the pharmaceutical composition comprises 0.54% moxifloxacin. In some embodiments, the pharmaceutical composition comprises 0.55% moxifloxacin.

In some embodiments, the pharmaceutical composition comprises bromfenac or a pharmaceutically acceptable salt thereof. In some embodiments, bromfenac may be known as bromfenac, bromfenac sodium, and bromfenac ophthalmic solution. In some embodiments, the bromfenac may be known as bromfenac sodium sesquihydrate. In some embodiments, the bromfenac may be a non-steroidal anti-inflammatory drug (NSAID). In some embodiments, bromfenac may block prostaglandin synthesis through cyclooxygenase inhibition, demonstrating COX-2 preference with a lesser affinity for COX-1. In some embodiments, bromfenac may be used as an analgesic. In some embodiments, bromfenac may be used to treat ocular pain. In some embodiments, bromfenac may be used to treat ocular inflammation. In some embodiments, bromfenac may be used to treat, promote, and/or facilitate post eye surgery healing and/or health.

In some embodiments, the pharmaceutical composition comprises bromfenac in an amount of about 0.075% with respect to weight per volume. In some embodiments, the pharmaceutical composition comprises from 0.0675% to 0.0825% bromfenac. In some embodiments, the pharmaceutical composition comprises from 0.0675% to 0.0700% bromfenac. In some embodiments, the pharmaceutical composition comprises from 0.0700% to 0.0725% bromfenac. In some embodiments, the pharmaceutical composition comprises from 0.0725% to 0.0750% bromfenac. In some embodiments, the pharmaceutical composition comprises from 0.0750% to 0.0775% bromfenac. In some embodiments, the pharmaceutical composition comprises from 0.0775% to 0.0800% bromfenac. In some embodiments, the pharmaceutical composition comprises from 0.0800% to 0.0825% bromfenac. In some embodiments, the pharmaceutical composition comprises 0.0675% bromfenac. In some embodiments, the pharmaceutical composition comprises 0.0680% bromfenac. In some embodiments, the pharmaceutical composition comprises 0.0685% bromfenac. In some embodiments, the pharmaceutical composition comprises 0.0690% bromfenac. In some embodiments, the pharmaceutical composition comprises 0.0695% bromfenac. In some embodiments, the pharmaceutical composition comprises 0.0700% bromfenac. In some embodiments, the pharmaceutical composition comprises 0.0705% bromfenac. In some embodiments, the pharmaceutical composition comprises 0.0710% bromfenac. In some embodiments, the pharmaceutical composition comprises 0.0715% bromfenac. In some embodiments, the pharmaceutical composition comprises 0.0720% bromfenac. In some embodiments, the pharmaceutical composition comprises 0.0725% bromfenac. In some embodiments, the pharmaceutical composition comprises 0.0730% bromfenac. In some embodiments, the pharmaceutical composition comprises 0.0735% bromfenac. In some embodiments, the pharmaceutical composition comprises 0.0740% bromfenac. In some embodiments, the pharmaceutical composition comprises 0.0745% bromfenac. In some embodiments, the pharmaceutical composition comprises 0.0750% bromfenac. In some embodiments, the pharmaceutical composition comprises 0.0755% bromfenac. In some embodiments, the pharmaceutical composition comprises 0.0760% bromfenac. In some embodiments, the pharmaceutical composition comprises 0.0765% bromfenac. In some embodiments, the pharmaceutical composition comprises 0.0770% bromfenac. In some embodiments, the pharmaceutical composition comprises 0.0775% bromfenac. In some embodiments, the pharmaceutical composition comprises 0.0780% bromfenac. In some embodiments, the pharmaceutical composition comprises 0.0785% bromfenac. In some embodiments, the pharmaceutical composition comprises 0.0790% bromfenac. In some embodiments, the pharmaceutical composition comprises 0.0795% bromfenac. In some embodiments, the pharmaceutical composition comprises 0.0800% bromfenac. In some embodiments, the pharmaceutical composition comprises 0.0805% bromfenac. In some embodiments, the pharmaceutical composition comprises 0.0810% bromfenac. In some embodiments, the pharmaceutical composition comprises 0.0815% bromfenac. In some embodiments, the pharmaceutical composition comprises 0.0820% bromfenac. In some embodiments, the pharmaceutical composition comprises 0.0825% bromfenac.

In some embodiments, the pH of the pharmaceutical composition is about 8. In some embodiments, the pH of the pharmaceutical composition is greater than 8. In some embodiments, the pH of the pharmaceutical composition is 7.0. In some embodiments, the pH of the pharmaceutical composition is 7.1. In some embodiments, the pH of the pharmaceutical composition is 7.2. In some embodiments, the pH of the pharmaceutical composition is 7.3. In some embodiments, the pH of the pharmaceutical composition is 7.4. In some embodiments, the pH of the pharmaceutical composition is 7.5. In some embodiments, the pH of the pharmaceutical composition is 7.6. In some embodiments, the pH of the pharmaceutical composition is 7.7. In some embodiments, the pH of the pharmaceutical composition is 7.8. In some embodiments, the pH of the pharmaceutical composition is 7.9. In some embodiments, the pH of the pharmaceutical composition is 8.0. In some embodiments, the pH of the pharmaceutical composition is 8.1. In some embodiments, the pH of the pharmaceutical composition is 8.2. In some embodiments, the pH of the pharmaceutical composition is 8.3. In some embodiments, the pH of the pharmaceutical composition is 8.4. In some embodiments, the pH of the pharmaceutical composition is 8.5. In some embodiments, the pH of the pharmaceutical composition is 8.6. In some embodiments, the pH of the pharmaceutical composition is 8.7. In some embodiments, the pH of the pharmaceutical composition is 8.8. In some embodiments, the pH of the pharmaceutical composition is 8.9. In some embodiments, the pH of the pharmaceutical composition is 9.0.

In some embodiments, compounding the pharmaceutical composition comprising 0.5% Moxifloxacin HCl and 0.075% Bromfenac may comprise steps of: (step 101) prepping clean work area (e.g., cleaning and/or disinfecting); (step 102) using only sterilized and/or depyrogenated equipment; (step 103) weighing applicable APIs (e.g., Moxifloxacin HCl and Bromfenac) in a powder hood (with the 0.5% and 0.075% targets in mind); (step 104) dissolving weighed out API powders in sterile water (or SWFI) (with the 0.5% and 0.075% targets in mind); (step 105) testing and adjusting the pH to a target of >8 via use of sodium hydroxide and pH meter (calibrated); (step 106) qs (“quantity sufficient”) with the sterile water (or SWFI) with the 0.5% and 0.075% targets in mind; (step 107) transferring resulting solution to a compounding aseptic isolator (CAI); (step 108) sterile filtering (e.g., a 0.22 micron filter) the resulting solution to yield the pharmaceutical composition comprising 0.5% Moxifloxacin HCl and 0.075% Bromfenac; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant.

### Pharmaceutical Composition 4: Difluprednate 0.05%/Moxifloxacin HCl 0.5%/Bromfenac 0.075%

In some embodiments, the pharmaceutical composition comprises difluprednate about 0.05%, moxifloxacin HCl about 0.5%, and bromfenac about 0.075%.

In some embodiments, the pharmaceutical composition comprises difluprednate or a pharmaceutically acceptable salt thereof. In some embodiments, the pharmaceutical composition comprises difluprednate. In some embodiments, difluprednate may be prepared to meet USP monograph for difluprednate ophthalmic solution. In some embodiments, difluprednate may be an anti-inflammatory steroid. A mechanism of action for difluprednate may be inhibition of migration of polymorphonuclear leukocytes and capilla increase reversal. In some embodiments, difluprednate may be used for treating inflammation in the eye, at the eye, and/or around the eye via use of eye drops. In some embodiments, difluprednate may be used in preparation for ocular surgery. In some embodiments, difluprednate may be used during ocular surgery. In some embodiments, difluprednate may be used after ocular surgery.

In some embodiments, the pharmaceutical composition comprises difluprednate in an amount of about 0.05% with respect to weight per volume. In some embodiments, the pharmaceutical composition comprises from 0.045% to 0.055% difluprednate. In some embodiments, the pharmaceutical composition comprises from about 0.045% to 0.050% difluprednate. In some embodiments, the pharmaceutical composition comprises from about 0.050% to 0.055% difluprednate. In some embodiments, the pharmaceutical composition comprises 0.045% difluprednate. In some embodiments, the pharmaceutical composition comprises 0.046% difluprednate. In some embodiments, the pharmaceutical composition comprises 0.047% difluprednate. In some embodiments, the pharmaceutical composition comprises 0.048% difluprednate. In some embodiments, the pharmaceutical composition comprises 0.049% difluprednate. In some embodiments, the pharmaceutical composition comprises 0.050% difluprednate. In some embodiments, the pharmaceutical composition comprises 0.051% difluprednate. In some embodiments, the pharmaceutical composition comprises 0.052% difluprednate. In some embodiments, the pharmaceutical composition comprises 0.053% difluprednate. In some embodiments, the pharmaceutical composition comprises 0.054% difluprednate. In some embodiments, the pharmaceutical composition comprises 0.055% difluprednate.

In some embodiments, the pharmaceutical composition comprises moxifloxacin or a pharmaceutically acceptable salt thereof. In some embodiments, moxifloxacin HCl may be known as moxifloxacin or moxifloxacin hydrochloride. In some embodiments, moxifloxacin HCl may be a synthetic fluoroquinolone antibacterial agent. In some embodiments, moxifloxacin may be used in an ophthalmic solution. In some embodiments, moxifloxacin may be used for the treatment of bacterial conjunctivitis (i.e., pink eye). Mechanism of action of moxifloxacin HCl may be through inhibition of DNA gyrase and topoisomerase IV which may be required for some bacterial DNA replication, transcription, repair, and/or recombination.

In some embodiments, the pharmaceutical composition comprises moxifloxacin in an amount of about 0.5% with respect to weight per volume. In some embodiments, the pharmaceutical composition comprises from 0.45% to 0.55% moxifloxacin. In some embodiments, the pharmaceutical composition comprises from 0.45% to 0.50% moxifloxacin. In some embodiments, the pharmaceutical composition comprises from 0.50% to 0.55% moxifloxacin. In some embodiments, the pharmaceutical composition comprises 0.45% moxifloxacin. In some embodiments, the pharmaceutical composition comprises 0.46% moxifloxacin. In some embodiments, the pharmaceutical composition comprises 0.47% moxifloxacin. In some embodiments, the pharmaceutical composition comprises 0.48% moxifloxacin. In some embodiments, the pharmaceutical composition comprises 0.49% moxifloxacin. In some embodiments, the pharmaceutical composition comprises 0.50% moxifloxacin. In some embodiments, the pharmaceutical composition comprises 0.51% moxifloxacin. In some embodiments, the pharmaceutical composition comprises 0.52% moxifloxacin. In some embodiments, the pharmaceutical composition comprises 0.53% moxifloxacin. In some embodiments, the pharmaceutical composition comprises 0.54% moxifloxacin. In some embodiments, the pharmaceutical composition comprises 0.55% moxifloxacin.

In some embodiments, the pharmaceutical composition comprises bromfenac or a pharmaceutically acceptable salt thereof. In some embodiments, bromfenac may be known as bromfenac, bromfenac sodium, and bromfenac ophthalmic solution. In some embodiments, the bromfenac may be known as bromfenac sodium sesquihydrate. In some embodiments, the bromfenac may be a non-steroidal anti-inflammatory drug (NSAID). In some embodiments, bromfenac may block prostaglandin synthesis through cyclooxygenase inhibition, demonstrating COX-2 preference with a lesser affinity for COX-1. In some embodiments, bromfenac may be used as an analgesic. In some embodiments, bromfenac may be used to treat ocular pain. In some embodiments, bromfenac may be used to treat ocular inflammation. In some embodiments, bromfenac may be used to treat, promote, and/or facilitate post eye surgery healing and/or health.

In some embodiments, the pharmaceutical composition comprises bromfenac in an amount of about 0.075% with respect to weight per volume. In some embodiments, the pharmaceutical composition comprises from 0.0675% to 0.0825% bromfenac. In some embodiments, the pharmaceutical composition comprises from 0.0675% to 0.0700% bromfenac. In some embodiments, the pharmaceutical composition comprises from 0.0700% to 0.0725% bromfenac. In some embodiments, the pharmaceutical composition comprises from 0.0725% to 0.0750% bromfenac. In some embodiments, the pharmaceutical composition comprises from 0.0750% to 0.0775% bromfenac. In some embodiments, the pharmaceutical composition comprises from 0.0775% to 0.0800% bromfenac. In some embodiments, the pharmaceutical composition comprises from 0.0800% to 0.0825% moxifloxacin. In some embodiments, the pharmaceutical composition comprises 0.0675% bromfenac. In some embodiments, the pharmaceutical composition comprises 0.0680% bromfenac. In some embodiments, the pharmaceutical composition comprises 0.0685% bromfenac. In some embodiments, the pharmaceutical composition comprises 0.0690% bromfenac. In some embodiments, the pharmaceutical composition comprises 0.0695% bromfenac. In some embodiments, the pharmaceutical composition comprises 0.0700% bromfenac. In some embodiments, the pharmaceutical composition comprises 0.0705% bromfenac. In some embodiments, the pharmaceutical composition comprises 0.0710% bromfenac. In some embodiments, the pharmaceutical composition comprises 0.0715% bromfenac. In some embodiments, the pharmaceutical composition comprises 0.0720% bromfenac. In some embodiments, the pharmaceutical composition comprises 0.0725% bromfenac. In some embodiments, the pharmaceutical composition comprises 0.0730% bromfenac. In some embodiments, the pharmaceutical composition comprises 0.0735% bromfenac. In some embodiments, the pharmaceutical composition comprises 0.0740% bromfenac. In some embodiments, the pharmaceutical composition comprises 0.0745% bromfenac. In some embodiments, the pharmaceutical composition comprises 0.0750% bromfenac. In some embodiments, the pharmaceutical composition comprises 0.0755% bromfenac. In some embodiments, the pharmaceutical composition comprises 0.0760% bromfenac. In some embodiments, the pharmaceutical composition comprises 0.0765% bromfenac. In some embodiments, the pharmaceutical composition comprises 0.0770% bromfenac. In some embodiments, the pharmaceutical composition comprises 0.0775% bromfenac. In some embodiments, the pharmaceutical composition comprises 0.0780% bromfenac. In some embodiments, the pharmaceutical composition comprises 0.0785% bromfenac. In some embodiments, the pharmaceutical composition comprises 0.0790% bromfenac. In some embodiments, the pharmaceutical composition comprises 0.0795% bromfenac. In some embodiments, the pharmaceutical composition comprises 0.0800% bromfenac. In some embodiments, the pharmaceutical composition comprises 0.0805% bromfenac. In some embodiments, the pharmaceutical composition comprises 0.0810% bromfenac. In some embodiments, the pharmaceutical composition comprises 0.0815% bromfenac. In some embodiments, the pharmaceutical composition comprises 0.0820% bromfenac. In some embodiments, the pharmaceutical composition comprises 0.0825% bromfenac.

In some embodiments, the pH of the pharmaceutical composition is about 8. In some embodiments, the pH of the pharmaceutical composition is greater than 8. In some embodiments, the pH of the pharmaceutical composition is 7.0. In some embodiments, the pH of the pharmaceutical composition is 7.1. In some embodiments, the pH of the pharmaceutical composition is 7.2. In some embodiments, the pH of the pharmaceutical composition is 7.3. In some embodiments, the pH of the pharmaceutical composition is 7.4. In some embodiments, the pH of the pharmaceutical composition is 7.5. In some embodiments, the pH of the pharmaceutical composition is 7.6. In some embodiments, the pH of the pharmaceutical composition is 7.7. In some embodiments, the pH of the pharmaceutical composition is 7.8. In some embodiments, the pH of the pharmaceutical composition is 7.9. In some embodiments, the pH of the pharmaceutical composition is 8.0. In some embodiments, the pH of the pharmaceutical composition is 8.1. In some embodiments, the pH of the pharmaceutical composition is 8.2. In some embodiments, the pH of the pharmaceutical composition is 8.3. In some embodiments, the pH of the pharmaceutical composition is 8.4. In some embodiments, the pH of the pharmaceutical composition is 8.5. In some embodiments, the pH of the pharmaceutical composition is 8.6. In some embodiments, the pH of the pharmaceutical composition is 8.7. In some embodiments, the pH of the pharmaceutical composition is 8.8. In some embodiments, the pH of the pharmaceutical composition is 8.9. In some embodiments, the pH of the pharmaceutical composition is 9.0.

In some embodiments, compounding the pharmaceutical composition comprising 0.05% Difluprednate, 0.5% Moxifloxacin HCl, and 0.075% Bromfenac may comprise steps of: (step 101) prepping clean work area (e.g., cleaning and/or disinfecting); (step 102) using only sterilized and/or depyrogenated equipment; (step 103) weighing applicable APIs (e.g., Difluprednate, Moxifloxacin HCl, and Bromfenac) in a powder hood (with the 0.05%, 0.5%, and 0.075% targets in mind); (step 104) dissolving weighed out API powders in sterile water (or SWFI) (with the 0.05%, 0.5%, and 0.075% targets in mind); (step 105) testing and adjusting the pH to a target of >8 via use of sodium hydroxide and pH meter (calibrated); (step 106) qs (“quantity sufficient”) with the sterile water (or SWFI) with the 0.05%, 0.5%, and 0.075% targets in mind; (step 107) transferring resulting solution to ISO 5 area; (step 108) sterile filtering aqueous phase (e.g., a 0.22 micron filter), adding the lipophilic phase, and homogenizing to achieve an emulsion to yield the pharmaceutical composition comprising 0.05% Difluprednate, 0.5% Moxifloxacin HCl, and 0.075% Bromfenac; (step 109) QA/QC (quality assurance/quality control) tests, such as bubble point testing, sterility testing, and/or endotoxin testing; (step 110) and filling final delivery device, e.g., a sterile ophthalmic dropper bottle (e.g., a “drop-tainer,” “steri-dropper,” or the like); and (step 111) of label and storage. See e.g., FIG. 1. In some embodiments, the final delivery device, e.g., the sterile ophthalmic dropper bottle, may be light resistant. In some embodiments the final delivery device with drug may be terminally sterilized.

### Aqueous Solution Stability

In some embodiments, the pharmaceutical composition described herein comprises a buffer. In some embodiments, a buffer is selected from borates, borate-polyol complexes, phosphate buffering agents, citrate buffering agents, acetate buffering agents, carbonate buffering agents, organic buffering agents, amino acid buffering agents, or combinations thereof.

In some embodiments, borates include boric acid, salts of boric acid, other pharmaceutically acceptable borates, and combinations thereof. In some embodiments, borates include boric acid, sodium borate, potassium borate, calcium borate, magnesium borate, manganese borate, and other such borate salts.

As used herein, the term “polyol” includes any compound having at least one hydroxyl group on each of two adjacent carbon atoms that are not in trans configuration relative to each other. In some embodiments, a polyols is linear or cyclic, substituted or unsubstituted, or mixtures thereof, so long as the resultant complex is water soluble and pharmaceutically acceptable. In some embodiments, examples of polyol include: sugars, sugar alcohols, sugar acids, and uronic acids. In some embodiments, polyols include but are not limited to mannitol, glycerin, xylitol, and sorbitol.

In some embodiments, phosphate buffering agents include phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; ammonium phosphates such as diammonium hydrogen phosphate and ammonium dihydrogen phosphate; or a combination thereof.

In some embodiments, the phosphate buffering agent is an anhydride. In some embodiments, the phosphate buffering agent is a hydrate.

In some embodiments, borate-polyol complexes include those described in U.S. Pat. No. 6,503,497.

In some embodiments, citrate buffering agents include citric acid and sodium citrate. In some embodiments, the citrate buffering agent comprises citrate.

In some embodiments, acetate buffering agents include acetic acid, potassium acetate, and sodium acetate.

In some embodiments, carbonate buffering agents include sodium bicarbonate and sodium carbonate.

In some embodiments, organic buffering agents include Good's Buffer, such as for example 2-(N-morpholino)ethanesulfonic acid (MES), N-(2-Acetamido)iminodiacetic acid, N-(Carbamoylmethyl)iminodiacetic acid (ADA), piperazine-N,N′-bis(2-ethanesulfonic acid (PIPES), N-(2-acetamido)-2-aminoethanesulfonic acid (ACES), β-Hydroxy-4-morpholinepropanesulfonic acid, 3-Morpholino-2-hydroxypropanesulfonic acid (MOPSO), cholamine chloride, 3-(N-morpholino)propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propanesulfonic acid (TAPSO), piperazine-1,4-bis (2-hydroxypropanesulphonic acid) (POPSO), 4-(2-hydroxyethyl)piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; and diethanolamine (DEA).

In some embodiments, amino acid buffering agents include taurine, aspartic acid and its salts (e.g., potassium salts, etc), E-aminocaproic acid, and the like.

Provided herein, in some embodiments, is a pharmaceutical composition essentially free of a citrate buffering agent, an acetate buffering agent, or a combination thereof. In some embodiments, the pharmaceutical composition is substantially free of a citrate buffering agent, an acetate buffering agent, or a combination thereof. In some embodiments, the pharmaceutical composition has no detectable amount of a citrate buffering agent, an acetate buffering agent, or a combination thereof.

In some embodiments, the pharmaceutical composition described herein further comprises a pH adjusting agent. In some embodiments, the pH adjusting agent used is an acid or a base. In some embodiments, the base is selected from oxides, hydroxides, carbonates, bicarbonates, and the likes. In some embodiments, the oxides are metal oxides such as calcium oxide, magnesium oxide, and the likes; hydroxides are of alkali metals and alkaline earth metals such as sodium hydroxide, potassium hydroxide, calcium hydroxide, and the like; and carbonates are sodium carbonate, sodium bicarbonates, potassium bicarbonates, and the like. In some embodiments, the acid is a mineral acid or an organic acid such as hydrochloric acid, nitric acid, phosphoric acid, acetic acid, citric acid, fumaric acid, malic acid, tartaric acid, and the like. In some embodiments, the pH adjusting agent includes, but is not limited to, acetate, bicarbonate, ammonium chloride, citrate, phosphate, pharmaceutically acceptable salts thereof, and combinations or mixtures thereof. In some embodiments, the pH adjusting agent comprises HCl, NaOH, or combinations thereof.

In some embodiments, the pharmaceutical composition has a pH of between about 6 and about 9, about 6.5 to about 8.9, about 7.0 and about 8.8, about 7 and about 8.5, or about 7.5 and about 8. In some embodiments, the pharmaceutical composition has a pH of about 8.0. In some embodiments, the pharmaceutical composition has a pH of about 8.1. In some embodiments, the pharmaceutical composition has a pH of about 8.2. In some embodiments, the pharmaceutical composition has a pH of about 8.3. In some embodiments, the pharmaceutical composition has a pH of about 8.4. In some embodiments, the pharmaceutical composition has a pH of greater than about 6.5. In some embodiments, the pharmaceutical composition has a pH of greater than about 6.6. In some embodiments, the pharmaceutical composition has a pH of greater than about 6.7. In some embodiments, the pharmaceutical composition has a pH of greater than about 6.8. In some embodiments, the pharmaceutical composition has a pH of greater than about 6.9. In some embodiments, the pharmaceutical composition has a pH of greater than about 7.0. In some embodiments, the pharmaceutical composition has a pH of greater than about 7.1. In some embodiments, the pharmaceutical composition has a pH of greater than about 7.2. In some embodiments, the pharmaceutical composition has a pH of greater than about 7.3. In some embodiments, the pharmaceutical composition has a pH of greater than about 7.4. In some embodiments, the pharmaceutical composition has a pH of greater than about 7.5. In some embodiments, the pharmaceutical composition has a pH of greater than about 7.6. In some embodiments, the pharmaceutical composition has a pH of greater than about 7.7. In some embodiments, the pharmaceutical composition has a pH of greater than about 7.8. In some embodiments, the pharmaceutical composition has a pH of greater than about 7.9. In some embodiments, the pharmaceutical composition has a pH of greater than about 8.0. In some embodiments, the pharmaceutical composition has a pH of greater than about 8.1. In some embodiments, the pharmaceutical composition has a pH of greater than about 8.2. In some embodiments, the pharmaceutical composition has a pH of greater than about 8.3. In some embodiments, the pharmaceutical composition has a pH of greater than about 8.4. In some embodiments, the pharmaceutical composition has a pH of greater than about 8.5. In some embodiments, the pharmaceutical composition has a pH of greater than about 8.6. In some embodiments, the pharmaceutical composition has a pH of greater than about 8.7. In some embodiments, the pharmaceutical composition has a pH of greater than about 8.8. In some embodiments, the pharmaceutical composition has a pH of greater than about 8.9. In some embodiments, the pharmaceutical composition has a pH of greater than about 9.0. In some embodiments, the pH is the pH of the pharmaceutical composition after an extended period of time under a storage condition.

In some embodiments, the pharmaceutical composition has an initial pH of between about 6 and about 9, about 6.5 to about 8.9, about 7.0 and about 8.8, about 7 and about 8.5, or about 7.5 and about 8. In some embodiments, the pharmaceutical composition has an initial pH of about 8.0. In some embodiments, the pharmaceutical composition has an initial pH of about 8.1. In some embodiments, the pharmaceutical composition has an initial pH of about 8.2. In some embodiments, the pharmaceutical composition has an initial pH of about 8.3. In some embodiments, the pharmaceutical composition has an initial pH of about 8.4. In some embodiments, the pharmaceutical composition has an initial pH of about 8.5. In some embodiments, the pharmaceutical composition has an initial pH of greater than about 6.5. In some embodiments, the pharmaceutical composition has an initial pH of greater than about 6.6. In some embodiments, the pharmaceutical composition has an initial pH of greater than about 6.7. In some embodiments, the pharmaceutical composition has an initial pH of greater than about 6.8. In some embodiments, the pharmaceutical composition has an initial pH of greater than about 6.9. In some embodiments, the pharmaceutical composition has an initial pH of greater than about 7.0. In some embodiments, the pharmaceutical composition has an initial pH of greater than about 7.1. In some embodiments, the pharmaceutical composition has an initial pH of greater than about 7.2. In some embodiments, the pharmaceutical composition has an initial pH of greater than about 7.3. In some embodiments, the pharmaceutical composition has an initial pH of greater than about 7.4. In some embodiments, the pharmaceutical composition has an initial pH of greater than about 7.5. In some embodiments, the pharmaceutical composition has an initial pH of greater than about 7.6. In some embodiments, the pharmaceutical composition has an initial pH of greater than about 7.7. In some embodiments, the pharmaceutical composition has an initial pH of greater than about 7.8. In some embodiments, the pharmaceutical composition has an initial pH of greater than about 7.9. In some embodiments, the pharmaceutical composition has an initial pH of greater than about 8.0. In some embodiments, the pharmaceutical composition has an initial pH of greater than about 8.1. In some embodiments, the pharmaceutical composition has an initial pH of greater than about 8.2. In some embodiments, the pharmaceutical composition has an initial pH of greater than about 8.3. In some embodiments, the pharmaceutical composition has an initial pH of greater than about 8.4. In some embodiments, the pharmaceutical composition has an initial pH of greater than about 8.5. In some embodiments, the pharmaceutical composition has an initial pH of greater than about 8.6. In some embodiments, the pharmaceutical composition has an initial pH of greater than about 8.7. In some embodiments, the pharmaceutical composition has an initial pH of greater than about 8.8. In some embodiments, the pharmaceutical composition has an initial pH of greater than about 8.9. In some embodiments, the pharmaceutical composition has an initial pH of greater than about 9.0. In some embodiments, the pH is the pH of the pharmaceutical composition after an extended period of time under a storage condition.

In some embodiments, the pH of the pharmaceutical composition described herein is associated with the stability of the pharmaceutical composition. In some embodiments, a stable pharmaceutical composition has a pH of between about 6 and about 9, about 6.5 to about 8.9, about 7.0 and about 8.8, about 7 and about 8.5, or about 7.5 and about 8. In some embodiments, a stable pharmaceutical composition has a pH of about 8.0. In some embodiments, a stable pharmaceutical composition has a pH of about 8.1. In some embodiments, a stable pharmaceutical composition has a pH of about 8.2. In some embodiments, a stable pharmaceutical composition has a pH of about 8.3. In some embodiments, a stable pharmaceutical composition has a pH of about 8.4. In some embodiments, a stable pharmaceutical composition has a pH of about 8.5. In some embodiments, a stable pharmaceutical composition has a pH of greater than about 6.5. In some embodiments, a stable pharmaceutical composition has a pH of greater than about 6.6. In some embodiments, a stable pharmaceutical composition has a pH of greater than about 6.7. In some embodiments, a stable pharmaceutical composition has a pH of greater than about 6.8. In some embodiments, a stable pharmaceutical composition has a pH of greater than about 6.9. In some embodiments, a stable pharmaceutical composition has a pH of greater than about 7.0. In some embodiments, a stable pharmaceutical composition has a pH of greater than about 7.1. In some embodiments, a stable pharmaceutical composition has a pH of greater than about 7.2. In some embodiments, a stable pharmaceutical composition has a pH of greater than about 7.3. In some embodiments, a stable pharmaceutical composition has a pH of greater than about 7.4. In some embodiments, a stable pharmaceutical composition has a pH of greater than about 7.5. In some embodiments, a stable pharmaceutical composition has a pH of greater than about 7.6. In some embodiments, a stable pharmaceutical composition has a pH of greater than about 7.7. In some embodiments, a stable pharmaceutical composition has a pH of greater than about 7.8. In some embodiments, a stable pharmaceutical composition has a pH of greater than about 7.9. In some embodiments, a stable pharmaceutical composition has a pH of greater than about 8.0. In some embodiments, a stable pharmaceutical composition has a pH of greater than about 8.1. In some embodiments, a stable pharmaceutical composition has a pH of greater than about 8.2. In some embodiments, a stable pharmaceutical composition has a pH of greater than about 8.3. In some embodiments, a stable pharmaceutical composition has a pH of greater than about 8.4. In some embodiments, a stable pharmaceutical composition has a pH of greater than about 8.5. In some embodiments, a stable pharmaceutical composition has a pH of greater than about 8.6. In some embodiments, a stable pharmaceutical composition has a pH of greater than about 8.7. In some embodiments, a stable pharmaceutical composition has a pH of greater than about 8.8. In some embodiments, a stable pharmaceutical composition has a pH of greater than about 8.9. In some embodiments, a stable pharmaceutical composition has a pH of greater than about 9.0.

The pharmaceutical composition described herein, in some embodiments, is substantially free of a preservative. In some embodiments, the pharmaceutical composition is substantially free of a benzalkonium chloride preservative. In some embodiments, the pharmaceutical composition has no detectable amount of a benzalkonium chloride preservative. In some embodiments, the pharmaceutical composition has no detectable amount of a benzalkonium chloride. In some embodiments, the pharmaceutical composition is substantially free of a preservative selected from cetrimonium, sodium perborate, stabilized oxychloro complex, SofZia, polyquaternium-1, chlorobutanol, edetate disodium, polyhexamethylene biguanide, or combinations thereof. In some embodiments, the pharmaceutical composition has no detectable amount of a preservative. In some embodiments, the pharmaceutical composition is substantially free of any preservative.

In some embodiments, the pharmaceutical composition described herein is stored in a plastic container. In some embodiments, the material of the plastic container comprises high density polyethylene (HDPE), low density polyethylene (LDPE), polyethylene terephthalate (PET), polyvinyl chloride (PVC), polypropylene (PP), polystyrene (PS), fluorine treated HDPE, post-consumer resin (PCR), K-resin (SBC), or bioplastic. In some embodiments, the material of the plastic container comprises LDPE.

In some embodiments, the pharmaceutical composition described herein is stored in a plastic container. In some embodiments, the pharmaceutical composition stored in a plastic container has a pH of between about 6 and about 9, about 6.5 to about 8.9, about 7.0 and about 8.8, about 7 and about 8.5, or about 7.5 and about 8. In some embodiments, the pharmaceutical composition stored in a plastic container has a pH of about 8.0. In some embodiments, the pharmaceutical composition stored in a plastic container has a pH of about 8.1. In some embodiments, the pharmaceutical composition stored in a plastic container has a pH of about 8.2. In some embodiments, the pharmaceutical composition stored in a plastic container has a pH of about 8.3. In some embodiments, the pharmaceutical composition stored in a plastic container has a pH of about 8.4. In some embodiments, the pharmaceutical composition stored in a plastic container has a pH of about 8.5. In some embodiments, the pharmaceutical composition stored in a plastic container has a pH of greater than about 6.5. In some embodiments, the pharmaceutical composition stored in a plastic container has a pH of greater than about 6.6. In some embodiments, the pharmaceutical composition stored in a plastic container has a pH of greater than about 6.7. In some embodiments, the pharmaceutical composition stored in a plastic container has a pH of greater than about 6.8. In some embodiments, the pharmaceutical composition stored in a plastic container has a pH of greater than about 6.9. In some embodiments, the pharmaceutical composition stored in a plastic container has a pH of greater than about 7.0. In some embodiments, the pharmaceutical composition stored in a plastic container has a pH of greater than about 7.1. In some embodiments, the pharmaceutical composition stored in a plastic container has a pH of greater than about 7.2. In some embodiments, the pharmaceutical composition stored in a plastic container has a pH of greater than about 7.3. In some embodiments, the pharmaceutical composition stored in a plastic container has a pH of greater than about 7.4. In some embodiments, the pharmaceutical composition stored in a plastic container has a pH of greater than about 7.5. In some embodiments, the pharmaceutical composition stored in a plastic container has a pH of greater than about 7.6. In some embodiments, the pharmaceutical composition stored in a plastic container has a pH of greater than about 7.7. In some embodiments, the pharmaceutical composition stored in a plastic container has a pH of greater than about 7.8. In some embodiments, the pharmaceutical composition stored in a plastic container has a pH of greater than about 7.9. In some embodiments, the pharmaceutical composition stored in a plastic container has a pH of greater than about 8.0. In some embodiments, the pharmaceutical composition stored in a plastic container has a pH of greater than about 8.1. In some embodiments, the pharmaceutical composition stored in a plastic container has a pH of greater than about 8.2. In some embodiments, the pharmaceutical composition stored in a plastic container has a pH of greater than about 8.3. In some embodiments, the pharmaceutical composition stored in a plastic container has a pH of greater than about 8.4. In some embodiments, the pharmaceutical composition stored in a plastic container has a pH of greater than about 8.5. In some embodiments, the pharmaceutical composition stored in a plastic container has a pH of greater than about 8.6. In some embodiments, the pharmaceutical composition stored in a plastic container has a pH of greater than about 8.7. In some embodiments, the pharmaceutical composition stored in a plastic container has a pH of greater than about 8.8. In some embodiments, the pharmaceutical composition stored in a plastic container has a pH of greater than about 8.9. In some embodiments, the pharmaceutical composition has a pH of greater than about 9.0.

In some embodiments, the pharmaceutical composition stored in a plastic container has a potency of at least 80% after an extended period of time under a storage condition. In some embodiments, the pharmaceutical composition stored in a plastic container has a potency of at least 85% after an extended period of time under a storage condition. In some embodiments, the pharmaceutical composition stored in a plastic container has a potency of at least 90% after an extended period of time under a storage condition. In some embodiments, the pharmaceutical composition stored in a plastic container has a potency of at least 93% after an extended period of time under a storage condition. In some embodiments, the pharmaceutical composition stored in a plastic container has a potency of at least 95% after an extended period of time under a storage condition. In some embodiments, the pharmaceutical composition stored in a plastic container has a potency of at least 97% after an extended period of time under a storage condition. In some embodiments, the pharmaceutical composition stored in a plastic container has a potency of at least 98% after an extended period of time under a storage condition. In some embodiments, the pharmaceutical composition stored in a plastic container has a potency of at least 99% after an extended period of time under a storage condition. In some embodiments, the storage condition comprises a temperature of about 25° C., about 40° C., or about 60° C. In some embodiments, the extended period of time is at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months.

In some embodiments, the pharmaceutical composition stored in a plastic container has a potency of at least 80% at a temperature of about 0° C., about 2° C., about 5° C., about 10° C., about 15° C., about 25° C., about 40° C., or about 60° C. In some embodiments, the pharmaceutical composition stored in a plastic container has a potency of at least 85% at a temperature of about 0° C., about 2° C., about 5° C., about 10° C., about 15° C., about 25° C., about 40° C., or about 60° C. In some embodiments, the pharmaceutical composition stored in a plastic container has a potency of at least 90% at a temperature of about 0° C., about 2° C., about 5° C., about 10° C., about 15° C., about 25° C., about 40° C., or about 60° C. In some embodiments, the pharmaceutical composition stored in a plastic container has a potency of at least 93% at a temperature of about 0° C., about 2° C., about 5° C., about 10° C., about 15° C., about 25° C., about 40° C., or about 60° C. In some embodiments, the pharmaceutical composition stored in a plastic container has a potency of at least 95% at a temperature of about 0° C., about 2° C., about 5° C., about 10° C., about 15° C., about 25° C., about 40° C., or about 60° C. In some embodiments, the pharmaceutical composition stored in a plastic container has a potency of at least 97% at a temperature of about 0° C., about 2° C., about 5° C., about 10° C., about 15° C., about 25° C., about 40° C., or about 60° C. In some embodiments, the pharmaceutical composition stored in a plastic container has a potency of at least 98% at a temperature of about 0° C., about 2° C., about 5° C., about 10° C., about 15° C., about 25° C., about 40° C., or about 60° C. In some embodiments, the pharmaceutical composition stored in a plastic container has a potency of at least 99% at a temperature of about 0° C., about 2° C., about 5° C., about 10° C., about 15° C., about 25° C., about 40° C., or about 60° C. In some embodiments, the pharmaceutical composition stored in a plastic container has a potency of at least 80%, at least 85%, at least 90%, at least 93%, at least 95%, at least 97%, at least 98%, or at least 99% at a temperature of from about 0° C. to about 30° C., 2° C. to about 10° C. or from about 16° C. to about 26° C.

In some embodiments, the pharmaceutical composition stored in a plastic container has a potency of at least 80% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 85% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 90% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 93% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 95% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 97% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 98% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months. In some embodiments, the pharmaceutical composition stored in a plastic container has a potency of at least 99% for a period of at least 1 week, at least 2 weeks, at least 3 weeks, at least 1 month, at least 2 months, at least 3 months, at least 4 months, at least 5 months, at least 6 months, at least 8 months, at least 10 months, at least 12 months, at least 18 months, or at least 24 months.

In some embodiments, the pharmaceutical composition described herein is formulated as an aqueous solution. In some embodiments, the aqueous solution is a stable aqueous solution. In some embodiments, the aqueous solution is stored in a plastic container as described above. In some embodiments, the aqueous solution is not stored in a glass container. In some embodiments, the aqueous solution is stored in the dark. In some embodiments, the aqueous solution is stored in the presence of light. In some embodiments, the aqueous solution is stable in the presence of light.

In some embodiments, the ophthalmically acceptable pharmaceutical formulations described herein are stable with respect to compound degradation (e.g. less than 30% degradation, less than 25% degradation, less than 20% degradation, less than 15% degradation, less than 10% degradation, less than 8% degradation, less than 5% degradation, less than 3% degradation, less than 2% degradation, or less than 5% degradation) over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months under storage conditions (e.g. room temperature). In other embodiments, the formulations described herein are stable with respect to compound degradation over a period of at least about 1 week. Also described herein are formulations that are stable with respect to compound degradation over a period of at least about 1 month.

### Aqueous Solution Dose-to-Dose Uniformity

Typically, ophthalmic aqueous solutions are packaged in eye drop bottles and administered as drops. For example, a single administration (i.e. a single dose) of an ophthalmic aqueous solution includes a single drop, two drops, three drops, or more into the eyes of the patient. In some embodiments, one dose of the ophthalmic aqueous solution described herein is one drop of the aqueous solution composition from the eye drop bottle.

In some embodiments, described herein are ophthalmic pharmaceutical compositions which provide a dose-to-dose uniform concentration. In some embodiments, the dose-to-dose uniform concentration does not present significant variations of drug content from one dose to another. In some embodiments, the dose-to-dose uniform concentration does provide consistent drug content from one dose to another.

In some embodiments, the pharmaceutical composition has a dose-to-dose ophthalmic agent concentration variation of less than 50%. In some embodiments, the pharmaceutical composition has a dose-to-dose ophthalmic agent concentration variation of less than 40%. In some embodiments, the pharmaceutical composition has a dose-to-dose ophthalmic agent concentration variation of less than 30%. In some embodiments, the pharmaceutical composition has a dose-to-dose ophthalmic agent concentration variation of less than 20%. In some embodiments, the pharmaceutical composition has a dose-to-dose ophthalmic agent concentration variation of less than 10%. In some embodiments, the pharmaceutical composition has a dose-to-dose ophthalmic agent concentration variation of less than 5%.

In some embodiments, the dose-to-dose ophthalmic agent concentration variation is based on 10 consecutive doses. In some embodiments, the dose-to-dose ophthalmic agent concentration variation is based on 8 consecutive doses. In some embodiments, the dose-to-dose ophthalmic agent concentration variation is based on 5 consecutive doses. In some embodiments, the dose-to-dose ophthalmic agent concentration variation is based on 3 consecutive doses. In some embodiments, the dose-to-dose ophthalmic agent concentration variation is based on 2 consecutive doses.

### Sterility

In some embodiments, the pharmaceutical compositions are sterilized. Included within the embodiments disclosed herein are means and processes for sterilization of a pharmaceutical composition disclosed herein for use in humans. The U. S. Food and Drug Administration has provided regulatory guidance in the publication “Guidance for Industry: Sterile Drug Products Produced by Aseptic Processing” available at: http://www.fda.gov/cder/guidance/5882fnl.htm, which is incorporated herein by reference in its entirety.

As used herein, sterilization means a process used to destroy or remove microorganisms that are present in a product or packaging. Any suitable method available for sterilization of objects and compositions is used. Available methods for the inactivation of microorganisms include, but are not limited to, the application of extreme heat, lethal chemicals, or gamma radiation. In some embodiments, a process for the preparation of an ophthalmic formulation comprises subjecting the formulation to a sterilization method selected from heat sterilization, chemical sterilization, radiation sterilization, or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter.

### Filtration

Filtration sterilization is a method used to remove but not destroy microorganisms from solutions. Membrane filters are used to filter heat-sensitive solutions. Such filters are thin, strong, homogenous polymers of mixed cellulosic esters (MCE), polyvinylidene fluoride (PVF; also known as PVDF), or polytetrafluoroethylene (PTFE) and have pore sizes ranging from 0.1 to 0.22 m. Solutions of various characteristics are optionally filtered using different filter membranes. For example, PVF and PTFE membranes are well suited to filtering organic solvents while aqueous solutions are filtered through PVF or MCE membranes. Filter apparatus are available for use on many scales ranging from the single point-of-use disposable filter attached to a syringe up to commercial scale filters for use in manufacturing plants. The membrane filters are sterilized by autoclave or chemical sterilization. Validation of membrane filtration systems is performed following standardized protocols (Microbiological Evaluation of Filters for Sterilizing Liquids, Vol 4, No. 3. Washington, D.C.: Health Industry Manufacturers Association, 1981) and involve challenging the membrane filter with a known quantity (ca. 10⁷/cm²) of unusually small microorganisms, such as Brevundimonas diminuta (ATCC 19146).

Pharmaceutical compositions are optionally sterilized by passing through membrane filters. In some embodiments, the methods disclosed herein comprise sterilizing the formulation (or components thereof) by means of filtration sterilization.

### Radiation Sterilization

One advantage of radiation sterilization is the ability to sterilize many types of products without heat degradation or other damage. The radiation commonly employed is beta radiation or alternatively, gamma radiation from a ⁶⁰Co source. The penetrating ability of gamma radiation allows its use in the sterilization of many product types, including solutions, compositions, and heterogeneous mixtures. The germicidal effects of irradiation arise from the interaction of gamma radiation with biological macromolecules. This interaction generates charged species and free-radicals. Subsequent chemical reactions, such as rearrangements and cross-linking processes, result in the loss of normal function for these biological macromolecules. The formulations described herein are also optionally sterilized using beta irradiation.

### Sterilization by Heat

Many methods are available for sterilization by the application of high heat. One method is through the use of a saturated steam autoclave. In this method, saturated steam at a temperature of at least 121° C. is allowed to contact the object to be sterilized. The transfer of heat is either directly to the microorganism, in the case of an object to be sterilized, or indirectly to the microorganism by heating the bulk of an aqueous solution to be sterilized. This method is widely practiced as it allows flexibility, safety, and economy in the sterilization process.

### Microorganisms

In some embodiments, the pharmaceutical compositions are substantially free of microorganisms. Acceptable bioburden or sterility levels are based on applicable standards that define therapeutically acceptable compositions. For example, acceptable sterility (e.g., bioburden) levels include about 10 colony forming units (cfu) per gram of formulation, about 50 cfu per gram of formulation, about 100 cfu per gram of formulation, about 500 cfu per gram of formulation or about 1000 cfu per gram of formulation. In some embodiments, acceptable bioburden levels or sterility for formulations include less than 10 cfu/mL, less than 50 cfu/mL, less than 500 cfu/mL or less than 1000 cfu/mL microbial agents. In addition, acceptable bioburden levels or sterility include the exclusion of specified objectionable microbiological agents. By way of example, specified objectionable microbiological agents include but are not limited to Escherichia coli (E. coli), Salmonella sp., Pseudomonas aeruginosa (P. aeruginosa) and/or other specific microbial agents.

An important component of the sterility assurance quality control, quality assurance, and validation process is the method of sterility testing. Sterility testing, by way of example only, is performed by two methods. The first is direct inoculation wherein a sample of the pharmaceutical composition to be tested is added to growth medium and incubated for a period of time up to 21 days. Turbidity of the growth medium indicates contamination. Drawbacks to this method include the small sampling size of bulk materials which reduces sensitivity, and detection of microorganism growth based on a visual observation. An alternative method is membrane filtration sterility testing. In this method, a volume of product is passed through a small membrane filter paper. The filter paper is then placed into media to promote the growth of microorganisms. This method has the advantage of greater sensitivity as the entire bulk product is sampled. The commercially available Millipore Steritest sterility testing system is optionally used for determinations by membrane filtration sterility testing.

Testing for E. coli and Salmonella includes the use of lactose broths incubated at 30-35° C. for 24-72 hours, incubation in MacConkey and/or EMB agars for 18-24 hours, and/or the use of Rappaport medium. Testing for the detection of P. aeruginosa includes the use of NAC agar.

In certain embodiments, the ophthalmic pharmaceutical composition described herein has less than about 60 colony forming units (CFU), less than about 50 colony forming units, less than about 40 colony forming units, or less than about 30 colony forming units of microbial agents per gram of formulation. In certain embodiments, the ophthalmic pharmaceutical composition described herein is formulated to be isotonic with the eye.

### Endotoxins

An additional aspect of the sterilization process is the removal of by-products from the killing of microorganisms (hereinafter, “Product”). The process of depyrogenation removes pyrogens from the sample. Pyrogens are endotoxins or exotoxins which induce an immune response. An example of an endotoxin is the lipopolysaccharide (LPS) molecule found in the cell wall of gram-negative bacteria. While sterilization procedures such as autoclaving or treatment with ethylene oxide kill the bacteria, the LPS residue induces a proinflammatory immune response, such as septic shock. Because the molecular size of endotoxins varies widely, the presence of endotoxins is expressed in “endotoxin units” (EU). One EU is equivalent to 100 picograms of E. coli LPS. In some embodiments, humans develop a response to as little as 5 EU/kg of body weight. The bioburden (e.g., microbial limit) and/or sterility (e.g., endotoxin level) is expressed in any units as recognized in the art. In certain embodiments, ophthalmic pharmaceutical compositions described herein contain lower endotoxin levels (e.g. <4 EU/kg of body weight of a subject) when compared to conventionally acceptable endotoxin levels (e.g., 5 EU/kg of body weight of a subject). In some embodiments, the ophthalmic pharmaceutical composition has less than about 5 EU/kg of body weight of a subject. In other embodiments, the ophthalmic pharmaceutical composition has less than about 4 EU/kg of body weight of a subject. In additional embodiments, the ophthalmic pharmaceutical composition has less than about 3 EU/kg of body weight of a subject. In additional embodiments, the ophthalmic pharmaceutical composition has less than about 2 EU/kg of body weight of a subject.

In some embodiments, the ophthalmic pharmaceutical composition has less than about 5 EU/kg of pharmaceutical composition. In other embodiments, the ophthalmic pharmaceutical composition has less than about 4 EU/kg of pharmaceutical composition. In additional embodiments, the ophthalmic pharmaceutical composition has less than about 3 EU/kg of pharmaceutical composition. In other embodiments, the ophthalmic pharmaceutical composition has less than about 1 EU/kg of pharmaceutical composition. In additional embodiments, the ophthalmic pharmaceutical composition has less than about 0.2 EU/kg of pharmaceutical composition. In certain embodiments, ophthalmic pharmaceutical compositions described herein contain from about 1 to about 5 EU/mL of pharmaceutical composition. In certain embodiments, ophthalmic pharmaceutical compositions described herein contain from about 2 to about 5 EU/mL of pharmaceutical composition, from about 3 to about 5 EU/mL of pharmaceutical composition, or from about 4 to about 5 EU/mL of pharmaceutical composition.

In certain embodiments, ophthalmic pharmaceutical compositions described herein contain lower endotoxin levels (e.g. <0.5 EU/mL of pharmaceutical composition) when compared to conventionally acceptable endotoxin levels (e.g., 0.5 EU/mL of pharmaceutical composition). In some embodiments, the ophthalmic pharmaceutical composition has less than about 0.5 EU/mL of pharmaceutical composition. In other embodiments, the ophthalmic pharmaceutical composition has less than about 0.4 EU/mL of pharmaceutical composition. In additional embodiments, the ophthalmic pharmaceutical composition has less than about 0.2 EU/mL of pharmaceutical composition.

Pyrogen detection, by way of example only, is performed by several methods. Suitable tests for sterility include tests described in United States Pharmacopoeia (USP)<71> Sterility Tests (23rd edition, 1995). The rabbit pyrogen test and the Limulus amebocyte lysate test are both specified in the United States Pharmacopeia Chapters <85> and <151> (USP23/NF 18, Biological Tests, The United States Pharmacopeial Convention, Rockville, Md., 1995). Alternative pyrogen assays have been developed based upon the monocyte activation-cytokine assay. Uniform cell lines suitable for quality control applications have been developed and have demonstrated the ability to detect pyrogenicity in samples that have passed the rabbit pyrogen test and the Limulus amebocyte lysate test (Taktak et al, J. Pharm. Pharmacol. (1990), 43:578-82). In an additional embodiment, the ophthalmic formulation is subject to depyrogenation. In a further embodiment, the process for the manufacture of the ophthalmic pharmaceutical composition comprises testing the pharmaceutical composition for pyrogenicity. In certain embodiments, the pharmaceutical compositions described herein are substantially free of pyrogens.

### Methods of Treatment

Provided herein, in one aspect, is a method for treating an ocular condition of an eye, comprising administering a pharmaceutical composition at, in, or around the eye via a delivery device and per a predetermined dosing regimen, wherein:

the pharmaceutical composition is free of preservatives;

the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;
   wherein these percentages are with respect to weight per volume; and
the ocular condition is glaucoma, care after cataract surgery, care after LASIK surgery, care for a retina of the eye after cataract surgery, care for a retina of the eye after retina surgery, in preparation for an intraocular procedure, or during the intraocular procedure.

Provided herein, in another aspect, is a method for treating an ocular condition of an eye, comprising administering a pharmaceutical composition at, in, or around the eye via a delivery device and per a predetermined dosing regimen; wherein the pharmaceutical composition comprises at least two active pharmaceutical ingredients compounded and stored in communication with each other; wherein the pharmaceutical composition is free of preservatives; wherein the method is more effective as compared against a preexisting method; wherein the preexisting method administers the at least two active pharmaceutical ingredients from at least two separate and different containers; and wherein the pharmaceutical composition comprises one of:
(1) prednisolone PO₄ about 1%, moxifloxacin HCl about 0.5%, and bromfenac about 0.075%;
(2) prednisolone PO₄ about 1% and moxifloxacin HCl about 0.5%;
(3) moxifloxacin HCl about 0.5% and bromfenac about 0.075%;
(4) difluprednate about 0.05%, moxifloxacin HCl about 0.5%, and bromfenac about 0.075%;

wherein these percentages are with respect to weight per volume.

In some embodiments, the ophthalmic pharmaceutical compositions described herein are for use in the treatment of glaucoma, care after cataract surgery, care after LASIK surgery, care for a retina of the eye after cataract surgery, care for a retina of the eye after retina surgery, in preparation for an intraocular procure, or during an intraocular procedure. In some embodiments, the ophthalmic pharmaceutical compositions described herein are for use in the treatment of glaucoma. In some embodiments, the ophthalmic pharmaceutical compositions described herein are for use in care after cataract surgery. In some embodiments, the ophthalmic pharmaceutical compositions described herein are for use in care after LASIK surgery. In some embodiments, the ophthalmic pharmaceutical compositions described herein are for use in care for a retina of the eye after cataract surgery. In some embodiments, the ophthalmic pharmaceutical compositions described herein are for use in care for a retina of the eye after retina surgery. In some embodiments, the ophthalmic pharmaceutical compositions described herein are for use in preparation for an intraocular procedure. In some embodiments, the ophthalmic pharmaceutical compositions described herein are for use during an intraocular procedure.

In some embodiments, the ophthalmic pharmaceutical compositions described herein are packaged in eye drop bottles and administered as drops. For example, a single administration (i.e. a single dose) of an ophthalmic pharmaceutical composition includes a single drop, two drops, three drops or more into the eyes of the patient. In some embodiments, one dose of the ophthalmic pharmaceutical composition described herein is one drop of the aqueous composition from the eye drop bottle.

In some embodiments, the ophthalmic pharmaceutical composition is formulated as an ophthalmic solution for treatment of glaucoma, care after cataract surgery, care after LASIK surgery, care for a retina of the eye after cataract surgery, care for a retina of the eye after retina surgery, in preparation for an intraocular procure, or during an intraocular procedure.

In some embodiments, the ophthalmic pharmaceutical composition is stored below room temperature prior to first use. In some embodiments, the ophthalmic pharmaceutical composition is stored at between about 2° C. to about 10° C. prior to first use. In some embodiments, the ophthalmic pharmaceutical composition is stored at about 2° C., about 3° C., about 4° C., about 5° C., about 6° C., about 7° C., about 8° C., about 9° C., or about 10° C. prior to first use. In some embodiments, the ophthalmic pharmaceutical composition is stored at between about 4° C. to about 8° C. prior to first use.

In some embodiments, the ophthalmic pharmaceutical composition is stored at room temperature after first use. In some embodiments, the ophthalmic pharmaceutical composition is stored at between about 16° C. to about 26° C. after to first use. In some embodiments, the ophthalmic pharmaceutical composition is stored at about 16° C., about 17° C., about 18° C., about 19° C., about 20° C., about 21° C., about 22° C., about 23° C., about 24° C., about 25° C., or about 26° C. after to first use.

In some embodiments, the ophthalmic pharmaceutical compositions are administered as follows: the lower lid of the eye to be administered is pulled down and a predetermined amount of the pharmaceutical composition (e.g. 1-3 drops) is applied to the inside of the eyelid. The ophthalmic tip of the dispensing mechanism does not touch any surface to avoid contamination and/or injury.

In some embodiments, the ophthalmic pharmaceutical composition is administered at predetermined time intervals over an extended period of time. In some embodiments, the ophthalmic pharmaceutical composition is administered once every day. In some embodiments, the ophthalmic pharmaceutical composition is administered every other day. In some embodiments, the ophthalmic pharmaceutical composition is administered over 1 week, 2 weeks, 1 month, 2 months, 3 months, 6 moths, 1 year, 2 years, 3 years, 4 years, 5 years, 6 years, 7 years, 8 years, 9 years, 10 years, 11 years, or 12-15 years.

In some embodiments, the ophthalmic pharmaceutical composition is administered once per day, twice per day, three times per day, once every other day, once per week, once every other week, or once monthly. In some embodiments, the ophthalmic pharmaceutical composition is administered once per day. In some embodiments, the ophthalmic pharmaceutical composition is administered twice per day. In some embodiments, the ophthalmic pharmaceutical composition is administered three times per day. In some embodiments, the ophthalmic pharmaceutical composition is administered once every other day. In some embodiments, the ophthalmic pharmaceutical composition is administered once per week. In some embodiments, the ophthalmic pharmaceutical composition is administered once every other week. In some embodiments, the ophthalmic pharmaceutical composition is administered once monthly.

In some embodiments, the ophthalmic pharmaceutical composition is administered in doses having a dose-to-dose ophthalmic agent concentration variation of less than 50%, less than 40%, less than 30%, less than 20%, less than 10%, or less than 5%.

The number of times a pharmaceutical composition is administered to an individual in need thereof depends on the discretion of a medical professional, the disorder, the severity of the disorder, and the individual's response to the pharmaceutical composition. In some embodiments, a pharmaceutical composition disclosed herein is administered once to an individual in need thereof with a mild acute condition. In some embodiments, a pharmaceutical composition disclosed herein is administered more than once to an individual in need thereof with a moderate or severe acute condition. In the case wherein the patient's condition does not improve, upon the doctor's discretion the administration of an ophthalmic agent is administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the ophthalmic agent is given continuously; alternatively, the dose of drug being administered is temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday is from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's ophthalmic condition has occurred, a maintenance ophthalmic agent dose is administered if necessary. Subsequently, the dosage or the frequency of administration, or both, is optionally reduced, as a function of the symptoms, to a level at which the improved disease, disorder or condition is retained. In certain embodiments, patients require intermittent treatment on a long-term basis upon any recurrence of symptoms.

The amount of ophthalmic agent that will correspond to such an amount will vary depending upon factors such as the particular compound, disease condition and its severity, according to the particular circumstances surrounding the case, including, e.g., the specific ophthalmic agent being administered, the route of administration, the condition being treated, the target area being treated, and the subject or host being treated. The desired dose is presented in a single dose or as divided doses administered simultaneously (or over a short period of time) or at appropriate intervals.

In some embodiments, the initial administration is a particular ophthalmic agent and the subsequent administration a different pharmaceutical composition or ophthalmic agent.

### Delivery Device

In certain embodiments, described herein is an ophthalmic product, which comprises a fluid-dispensing device comprising a reservoir and a dispensing tip fitted onto the reservoir, and the pharmaceutical composition described herein, wherein the pharmaceutical composition is dispensed from the dispensing tip into an eye of an individual in need thereof. In some embodiments, the pharmaceutical composition in the reservoir is substantially preservative-free.

In some embodiments, the ophthalmic product comprises a delivery device. In some embodiments, the delivery device is an eye dropper. In some embodiments, the eye dropper is a multidose eye dropper. In some embodiments, the multidose eye dropper is (i) a dropper bottle for dispensing predetermined metered quantities of liquid, the dropper bottle comprising a non-return position preventing the liquid from flowing back into the dropper bottle; or (ii) an Ophthalmic Squeeze Dispenser (OSD) comprising a sealing closure member that closes a dispenser orifice when the liquid present near the dispenser orifice is at a pressure less than a predetermined threshold. In some embodiments, the multidose eye dropper is a dropper bottle for dispensing predetermined metered quantities of liquid, the dropper bottle comprising a non-return position preventing the liquid from flowing back into the dropper bottle. In some embodiments, the multidose eye dropper is an Ophthalmic Squeeze Dispenser (OSD) comprising a sealing closure member that closes a dispenser orifice when the liquid present near the dispenser orifice is at a pressure less than a predetermined threshold. As used herein, the term “substantially preservative-free” or “substantially free of a preservative” refers to the pharmaceutical composition as having one of: less than about 1%, less than about 0.5%, less than about 0.4%, less than about 0.3%, less than about 0.2%, less than about 0.1%, less than about 0.01%, or less than about 0.001% of a preservative. In some embodiments, the term refers to the pharmaceutical composition as having 0% of a preservative, or preservative-free.

In some embodiments, the ophthalmic product comprises a fluid-dispensing device comprising a reservoir and a dispensing tip fitted onto the reservoir; and an ophthalmic composition comprising about 1% prednisolone PO₄, about 0.5% moxifloxacin HCl, and about 0.075% bromfenac in the reservoir; wherein the ophthalmic composition is dispensed from the dispensing tip into an eye of an individual in need thereof, and wherein the dispensed ophthalmic composition is substantially preservative-free.

In some embodiments, the ophthalmic product comprises a fluid-dispensing device comprising a reservoir and a dispensing tip fitted onto the reservoir; and an ophthalmic composition comprising about 1% prednisolone PO₄ and about 0.5% moxifloxacin HCl in the reservoir; wherein the ophthalmic composition is dispensed from the dispensing tip into an eye of an individual in need thereof, and wherein the dispensed ophthalmic composition is substantially preservative-free.

In some embodiments, the ophthalmic product comprises a fluid-dispensing device comprising a reservoir and a dispensing tip fitted onto the reservoir; and an ophthalmic composition comprising about 0.5% moxifloxacin HCl and about 0.075% bromfenac in the reservoir; wherein the ophthalmic composition is dispensed from the dispensing tip into an eye of an individual in need thereof, and wherein the dispensed ophthalmic composition is substantially preservative-free.

In some embodiments, the ophthalmic product comprises a fluid-dispensing device comprising a reservoir and a dispensing tip fitted onto the reservoir; and an ophthalmic composition comprising about 0.05% difluprednate, about 0.5% moxifloxacin HCl, and about 0.075% bromfenac in the reservoir; wherein the ophthalmic composition is dispensed from the dispensing tip into an eye of an individual in need thereof, and wherein the dispensed ophthalmic composition is substantially preservative-free.

In some embodiments, the reservoir comprises a polymeric material, for example, polyvinyl chloride (PVC) plastics or non-PVC plastics. In some embodiments, the material of the reservoir comprises high-density polyethylene (HDPE), low-density polyethylene (LDPE), polyethylene terephthalate (PET), polyvinyl chloride (PVC), polypropylene (PP), polystyrene (PS), fluorine treated HDPE, post-consumer resin (PCR), K-resin (SBC), or bioplastic. In some embodiments, the material of the reservoir comprises ethylene vinyl acetate (EVA) and block copolymers such as Kraton®. In some embodiments, the material of the reservoir comprises high-density polyethylene (HDPE). In some embodiments, the material of the reservoir comprises low-density polyethylene (LDPE). In some embodiments, the material of the reservoir comprises polyethylene terephthalate (PET). In some embodiments, the material of the reservoir comprises polypropylene (PP). In some embodiments, the material of the reservoir comprises polystyrene (PS). In some embodiments, the material of the reservoir comprises ethylene vinyl acetate (EVA).

In some embodiments, the reservoir further comprises a plasticizer. Exemplary plasticizer includes families of phthalate esters such as di-2-ethylhexylphthalate (DEHP), mono-(2-ethylhexyl) phthalate (MEHP), and triethylhexyltrimellitate (TEHTM); citrate esters such as acetyltri-n-hexyl citrate, acetyltri-n-(hexyl/octyl/decyl) citrate, acetyltri-n-(octyl/decyl) citrate, and n-butyryltri-n-hexyl citrate; and non-phthalate plasticizers such as TEHTM, di(isononyl) cyclohexane-1,2-dicarboxylate (DINCH), or n-butyryltri-n-hexyl citrate.

In some embodiments, the reservoir is at least partially elastically deformable so as to dispense the ophthalmic composition by pressing on the reservoir.

In some embodiments, the reservoir comprises glass.

In some embodiments, the reservoir stores multiple unit doses of the pharmaceutical composition described herein.

In some embodiments, the fluid-dispensing device described herein is a multi-dose fluid-dispensing device.

In some embodiments, the fluid-dispensing device described herein enables storage of a preservative-free or substantially preservative-free composition. In some embodiments, the fluid-dispensing device is a multi-dose preservative-free device.

In some embodiments, a fluid-dispensing device from Aptar Pharma (AptarGroup) is utilized for delivery of a composition described herein. In some embodiments, the pharmaceutical composition is preservative-free.

In some embodiments, a fluid-dispensing device from Nemera La Verpilliére S.A.S. is utilized for delivery of a composition described herein. In some embodiments, a fluid-dispensing device as described in U.S. Pat. Nos. 8,986,266 and/or 8,863,998 is utilized for delivery of a composition described herein. In some embodiments, the pharmaceutical composition is preservative-free.

In some embodiments, a fluid-dispensing device from CIS Pharma is utilized for delivery of a composition described herein. In some embodiments, the pharmaceutical composition is preservative-free.

In some embodiments, the fluid-dispensing device described herein optionally comprises an atomizer, a pump, or a mister. In such embodiments, a mechanical system such as a pump, a mister, or an atomizer is incorporated into the fluid-dispensing device to facilitate delivery of the pharmaceutical composition described herein and optionally to facilitate dose uniformity (e.g., between each administration, minimize excessive drug volume, and/or enhance droplet uniformity).

In additional embodiments, a mechanical system such as a pump, a mister, or an atomizer is incorporated into the fluid-dispensing device to enhance and/or optimize the amount of drug delivered to the eye.

In some embodiments, an atomizer and/or pump system from Aero Pump GMBH (Adelphi Healthcare Packaging) is utilized with the fluid-dispensing device and the pharmaceutical composition described herein. In some embodiments, a multiple-dosage fluid-dispensing device from Aero Pump GMBH is utilized for delivery of the pharmaceutical composition described herein. In some embodiments, a fluid-dispensing device as described in U.S. Pat. No. 10,155,243 and/or US Patent Publication No. 2015/076174 (Aero Pump GMBH) is utilized with the fluid-dispensing device and the pharmaceutical composition described herein.

In some embodiments, a fluid-dispensing device from Eyenovia, Inc. is utilized for delivery of the pharmaceutical composition described herein. In some embodiments, a fluid-dispensing device comprising one or more of a delivery system and/or component described in U.S. Pat. Nos. 9,539,604, 9,087,145, 9,463,486, or 8,684,980 are utilized for delivery of the pharmaceutical composition described herein.

In some embodiments, a fluid-dispensing device comprising one or more of a delivery system and/or component from Kedalion Therapeutics is utilized for delivery of the pharmaceutical composition described herein.

In some embodiments, a fluid-dispensing device comprising one or more of a delivery system and/or component from Aptar Pharma (e.g., a pump dispensing system) is utilized for delivery of the pharmaceutical composition described herein.

In some embodiments, the fluid-dispensing device optionally comprises an internal filter or membrane. In some embodiments, the internal filter or membrane is located within the fluid-dispensing device at a position capable of removing a microorganism and/or an endotoxin from the ophthalmic composition prior to dispensing the ophthalmic composition into the eye of the individual. In some embodiments, the internal filter or membrane is located at the junction connecting the dispensing tip to the reservoir. In other cases, the internal filter or membrane is located within the dispensing tip. In some embodiments, the ophthalmic composition is a preservative-free composition.

In some embodiments, the internal filter or membrane comprises cellulose acetate, cellulose nitrate, nylon, polyether sulfone (PES), polypropylene (PP), polyvinyl difluoride (PVDF), silicone, polycarbonate, or a combination thereof.

In some embodiments, the dispensed composition comprises one of: less than about 1%, less than about 0.5%, less than about 0.4%, less than about 0.3%, less than about 0.2%, less than about 0.1%, less than about 0.01%, less than about 0.001%, or less than about 0.0001% of a preservative. In some embodiments, the dispensed composition is preservative-free.

In some embodiments, the droplet volume dispensed from the fluid-dispensing device described herein is from about 0.1 μL to about 50 μL. In some embodiments, the droplet volume is one of: about 0.1 μL to about 40 μL, about 0.5 μL to about 30 μL, about 1 μL to about 30 μL, about 5 μL to about 20 μL, about 10 μL to about 20 μL, about 5 μL to about 40 μL, about 5 μL to about 30 μL, about 6 μL to about 8 μL, about 6 μL to about 7 μL, about 7 μL to about 8 μL, about 10 μL to about 40 μL, or about 10 μL to about 30 μL. In some embodiments, the droplet volume dispensed from the fluid-dispensing device described herein is about 0.1 μL, about 0.2 μL, about 0.3 μL, about 0.4 μL, about 0.5 μL, about 1 μL, about 5 μL, about 6 μL, about 7 μL, about 8 μL, about 9 μL, about 10 μL, about 20 μL, about 30 μL, about 40 μL, or about 50 μL.

In some embodiments, the linear size or diameter of the droplet when spherical is about 1 up to less than 100 microns. In some embodiments, the linear size or diameter of the droplet is about 20 to 100 microns, about 1 to 20 microns, 1-15 microns, 1-10 microns, 8-20 microns, 8-15 microns, 8-12 microns, or 1-5 microns. In the context of an aerosol or mist, the size of the droplet is, for example, 1-5 microns, 1-10 microns, less than 10 microns, greater than 10 microns, or up to 100 microns.

In some embodiments, the diameter of the droplet is calculated using the equation V=4πr³ where the diameter=2r.

In some embodiments, the fluid-dispensing device described herein facilitates at least 60%, 70%, 80%, 85%, 90%, 95%, or 99% of the ejected mass of a droplet deposited on the eye of an individual. In some embodiments, the fluid-dispensing device described herein facilitates at least 70% of the ejected mass of a droplet to be deposited on the eye of an individual. In some embodiments, the fluid-dispensing device described herein facilitates at least 80% of the ejected mass of a droplet to be deposited on the eye of an individual. In some embodiments, the fluid-dispensing device described herein facilitates at least 90% of the ejected mass of a droplet to be deposited on the eye of an individual. In some embodiments, the fluid-dispensing device described herein facilitates at least 95% of the ejected mass of a droplet to be deposited on the eye of an individual. In some embodiments, the fluid-dispensing device described herein facilitates at least 99% of the ejected mass of a droplet to be deposited on the eye of an individual.

### Kits/Articles of Manufacture

This disclosure also provides kits for treatment of glaucoma, care after cataract surgery, care after LASIK surgery, care for a retina of the eye after cataract surgery, care for a retina of the eye after retina surgery, in preparation for an intraocular procedure, or during the intraocular procedure.

Such kits generally will comprise one or more of the ophthalmic pharmaceutical compositions disclosed herein and instructions for using the kit. This disclosure also contemplates the use of one or more of the ophthalmic pharmaceutical compositions in the manufacture of medicaments for treating, abating, reducing, or ameliorating the symptoms of a disease, dysfunction, or disorder in a mammal, such as a human.

In some embodiments, kits include a carrier, package, or container that is compartmentalized to receive one or more containers such as vials, tubes, or bottles. In other embodiments, the containers are formed from a variety of materials such as glass or plastic.

The articles of manufacture provided herein contain packaging materials. Packaging materials for use in packaging pharmaceutical products are also presented herein. See, e.g., U.S. Pat. Nos. 5,323,907, 5,052,558 and 5,033,252. Examples of pharmaceutical packaging materials include, but are not limited to, dropper bottles, tubes, pumps, bags, vials, containers, syringes, bottles, and any packaging material suitable for a selected formulation and intended mode of administration and treatment. A wide array of ophthalmic pharmaceutical compositions provided herein are contemplated as are a variety of treatments for any disease, disorder, or condition that benefits by controlled release administration of an ophthalmic agent to the eye.

In some embodiments, a kit includes one or more additional containers, each with one or more of various materials (such as rinses, wipes, and/or devices) desirable from a commercial and user standpoint for use of a pharmaceutical composition described herein. Such materials also include labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself; a label is associated with a container when it is present within a receptacle or carrier that also holds the container, e.g., as a package insert. In other embodiments, a label is used to indicate that the contents are to be used for a specific therapeutic application. In yet another embodiment, a label also indicates directions for use of the contents, such as in the methods described herein.

In certain embodiments, the ophthalmic pharmaceutical compositions are presented in a dispenser device which contains one or more unit dosage forms containing a pharmaceutical composition provided herein. In a further embodiment, the dispenser device is accompanied by instructions for administration. In yet a further embodiment, the dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a pharmaceutical composition provided herein formulated in a compatible pharmaceutical carrier are also prepared, placed in an appropriate container, and labeled for treatment of an indicated condition.

### EXAMPLES

### Example 1: Preparation of Prednisolone PO₄ 1%/Moxifloxacin HCl 0.5%/Bromfenac 0.075%

To 1,060 mL sterile water for injection (SWFI) was added moxifloxacin hydrochloride (33.3 g, 76.1 mmol). SWFI was added to bring the solution to a weight of 3,520 g and the resulting suspension was stirred for 10 minutes until all solids were dissolved. The solution was adjusted to pH >7 with 10% sodium hydroxide (30 mL).

In a separate container, to 1,050 mL SWFI were sequentially added boric acid (82.01 g, 1,326 mmol), prednisolone sodium phosphate (75.63 g, 155.5 mmol), and 1,060 mL SWFI. The solution was adjusted to pH >7 with 10% sodium hydroxide (95 mL) and added to the moxifloxacin solution. The resulting solution was adjusted to pH >8 with 10% sodium hydroxide if needed. Bromfenac sodium (5.54 g, 15.6 mmol) was added with stirring, and SWFI was added to bring the solution to a weight of 6,400 g. The solution was adjusted to pH >8 with 10% NaOH, and SWFI was added to bring the solution to a final weight of 6,447.4 g.

### Example 2: Preparation of Prednisolone PO₄ 1%/Moxifloxacin HCl 0.5%

To 3,080 mL sterile water for injection (SWFI) was added moxifloxacin hydrochloride (29.2 g, 66.7 mmol), and the resulting suspension was stirred until all solids were dissolved. The solution was adjusted to pH 7 with 10% sodium hydroxide.

In a separate container, to 1,060 mL SWFI were sequentially added boric acid (70.63 g, 1,142 mmol) and prednisolone sodium phosphate (66.28 g, 136.3 mmol). The solution was adjusted to pH >7 with 10% sodium hydroxide and added to the moxifloxacin solution. SWFI was added to bring the solution to a weight of 5,550 g. The solution was adjusted to pH >7 with 10% NaOH, and SWFI was added to bring the solution to a final weight of 5,650.4 g.

### Example 3: Preparation of Moxifloxacin HCl 0.5%/Bromfenac 0.075%

To 1,060 mL sterile water for injection (SWFI) was added moxifloxacin hydrochloride (33.3 g, 76.1 mmol). SWFI (3,180 mL) was added and the resulting suspension was stirred until all solids were dissolved. Boric acid (108.3 g, 1,752 mmol) was added and SWFI was added to bring the solution to a weight of 6,000 g. The solution was adjusted to pH >8 with 10% sodium hydroxide. Bromfenac sodium (5.54 g, 15.6 mmol) was added with stirring, and the solution was adjusted to pH >8 with 10% NaOH. SWFI was added to bring the solution to a final weight of 6,447.4 g.

### Example 4: Preparation of Difluprednate 0.05%/Moxifloxacin HCl 0.5%/Bromfenac 0.075%

Castor oil (200 g) was added to difluprednate (2.0 g, 3.93 mmol) and the resulting suspension was stirred at 70° C. until transparent and sterilized in an oven.

In a separate container, to 1,060 mL sterile water for injection (SWFI) was added moxifloxacin hydrochloride (20.7 g, 47.3 mmol). SWFI (3,180 mL) was added and the resulting suspension was stirred until all solids were dissolved. Povidone (80.0 g) was added and the resulting suspension was stirred until all solids were dissolved. Edetate disodium (0.80 g, 2.38 mmol) was added and the resulting suspension was stirred until all solids were dissolved. Glycerin (80.0 g, 869 mmol) and polysorbate 80 NF (160.0 g, 122 mmol) were added and the solution was adjusted to pH >8 with 10% sodium hydroxide. Bromfenac (3.464 g, 10.4 mmol) was added and the resulting suspension was stirred until all solids were dissolved. The difluprednate solution was added, the resulting solution was adjusted to pH >8 with 10% sodium hydroxide, and SWFI was added to bring the solution to a final weight of 4,000.0 g. The resulting suspension was emulsified with an autoclave-sterilized homogenizer head at 8,000 RPM for 1 hour.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-19. (canceled)

20. A method for treating an ocular condition of an eye, comprising administering a pharmaceutical composition at, in, or around the eye via a delivery device and per a predetermined dosing regimen, wherein:
the pharmaceutical composition comprises:
(a) difluprednate or a pharmaceutically acceptable salt thereof, in an amount that corresponds to 0.045% to 0.055% of difluprednate,
(b) moxifloxacin or a pharmaceutically acceptable salt thereof, in an amount that corresponds to 0.45% to 0.55% of moxifloxacin, and
(c) bromfenac or a pharmaceutically acceptable salt thereof, in an amount that corresponds to 0.0675% to 0.0825% of bromfenac;
wherein these percentages are with respect to weight per volume;
the pharmaceutical composition is free of preservatives; and
the ocular condition is care after cataract surgery, care after LASIK surgery, care for a retina of the eye after cataract surgery, care for a retina of the eye after retina surgery, in preparation for an intraocular procedure, or during the intraocular procedure.

21. The method of claim 20, wherein the delivery device is an eye dropper.

22. The method of claim 21, wherein the eye dropper is a multidose eye dropper.

23. The method of claim 20, wherein the predetermined dosing regimen is once per day, twice per day, three times per day, once every other day, once per week, once every other week, or once monthly.

24. The method of claim 20, wherein the pharmaceutical composition comprises moxifloxacin hydrochloride.

25. The method of claim 20, wherein the pharmaceutical composition comprises bromfenac sodium.

26. The method of claim 20, wherein the pharmaceutical composition comprises:
(a) difluprednate or a pharmaceutically acceptable salt thereof, in an amount that corresponds to about 0.05% of difluprednate,
(b) moxifloxacin or a pharmaceutically acceptable salt thereof, in an amount that corresponds to about 0.5% of moxifloxacin, and
(c) bromfenac or a pharmaceutically acceptable salt thereof, in an amount that corresponds to about 0.075% of bromfenac;
wherein these percentages are with respect to weight per volume.

27. The method of claim 26, wherein the pharmaceutical composition comprises moxifloxacin hydrochloride.

28. The method of claim 26, wherein the pharmaceutical composition comprises bromfenac sodium.

29. The method of claim 20, wherein the pharmaceutical composition comprises an emulsion.

30. A method for treating an ocular condition of an eye, comprising administering a pharmaceutical composition at, in, or around the eye via a delivery device and per a predetermined dosing regimen; wherein
the pharmaceutical composition comprises the following active pharmaceutical ingredients:
(a) difluprednate or a pharmaceutically acceptable salt thereof, in an amount that corresponds to 0.045% to 0.055% of difluprednate,
(b) moxifloxacin or a pharmaceutically acceptable salt thereof, in an amount that corresponds to 0.45% to 0.55% of moxifloxacin, and
(c) bromfenac or a pharmaceutically acceptable salt thereof, in an amount that corresponds to 0.0675% to 0.0825% of bromfenac;
wherein these percentages are with respect to weight per volume;
the pharmaceutical composition is free of preservatives;
at least two of the active pharmaceutical ingredients are compounded and stored in communication with each other; and
the method is more effective as compared to a preexisting method, wherein the preexisting method administers the at least two active pharmaceutical ingredients from at least two separate and different containers.

31. The method of claim 30, wherein the delivery device is an eye dropper.

32. The method of claim 31, wherein the eye dropper is a multidose eye dropper.

33. The method of claim 30, wherein the predetermined dosing regimen is once per day, twice per day, three times per day, once every other day, once per week, once every other week, or once monthly.

34. The method of claim 30, wherein the pharmaceutical composition comprises moxifloxacin hydrochloride.

35. The method of claim 30, wherein the pharmaceutical composition comprises bromfenac sodium.

36. The method of claim 30, wherein the pharmaceutical composition comprises:
(a) difluprednate or a pharmaceutically acceptable salt thereof, in an amount that corresponds to about 0.05% of difluprednate,
(b) moxifloxacin or a pharmaceutically acceptable salt thereof, in an amount that corresponds to about 0.5% of moxifloxacin, and
(c) bromfenac or a pharmaceutically acceptable salt thereof, in an amount that corresponds to about 0.075% of bromfenac;
wherein these percentages are with respect to weight per volume.

37. The method of claim 36, wherein the pharmaceutical composition comprises moxifloxacin hydrochloride.

38. The method of claim 36, wherein the pharmaceutical composition comprises bromfenac sodium.

39. The method of claim 30, wherein the pharmaceutical composition comprises an emulsion.
